(19) United States
(12) Patent Application Publication
(10) Pub. No.: US 2026/0203378 A1
(43) Pub. Date: Jul. 16, 2026

(54) MEASUREMENT SYSTEM AND METHOD FOR TRAINING A MACHINE LEARNING MODEL THEREFOR

(71) Applicant: HUNBIOME CO., LTD., Seoul (KR)

(72) Inventors: Yongju AN, Seoul (KR); Kung AHN, Gunpo-si, Gyeonggi-do (KR); Boreum PARK, Bucheon-si, Gyeonggi-do (KR)

(73) Assignee: HUNBIOME CO., LTD., Seoul (KR)

(21) Appl. No.: 19/137,345

(22) PCT Filed: Dec. 19, 2023
(86) PCT No.: PCT/KR2023/020935

§ 371 Date: Jun. 10, 2025

(30) Foreign Application Priority Data
Dec. 28, 2022 (KR) 10-2022-0187399
Dec. 28, 2022 (KR) 10-2022-0187400
Dec. 28, 2022 (KR) 10-2022-0187401

(51) Int. Cl. G06F 18/2415; G01N 1/30; G01N 1/31; G01N 1/34; G06N 20/00

(52) U.S. Cl. CPC G06F 18/2415; G01N 1/31; G01N 1/34; G06N 20/00; G01N 2001/302

(57) ABSTRACT

The present invention relates to a skin condition measurement system, wherein the measurement system includes: a sub-device including a color sensor for receiving an optical signal; and a main device including a reception hole for forming a path for the optical signal incident to the color sensor and a slot to which a film for collecting oil of a user's skin is inserted, wherein the slot extends to the reception hole.

### TECHNICAL FIELD

The present invention relates to a measurement system and a method for training a machine learning model therefor, and more particularly, to a measurement system that provides information regarding skin condition and skin type, and a method for training a machine learning model for the same.

### BACKGROUND ART

With the increasing variety of cosmetic products, personalized cosmetics have been attracting attention. To achieve personalization, it is necessary to assess an individual's skin condition. For example, a questionnaire-based method such as the one proposed in The Skin Type Solution by Dr. Leslie Baumann of the United States may be used to determine skin type.

However, such determination methods based solely on simple questionnaires involve subjective judgments and do not consider age, which results in absolute judgments that are unsuitable for providing personalized solutions.

### DETAILED DESCRIPTION OF THE INVENTION

### Technical Problem

Embodiments of the present invention are intended to provide objective information on skin condition and skin type, taking into account the age group to which the subject belongs.

Furthermore, the embodiments aim to enable quick and easy acquisition of information necessary for assessing the skin condition.

The embodiments also aim to facilitate the training of a machine learning model for assessing skin condition.

The technical problems to be solved by the present invention are not limited to those described above, and other technical problems can be derived from the embodiments described below.

### Solution to Problem

According to various embodiments of the present invention, a method for training a machine learning model for identifying skin type, executed by a computing device, comprises: collecting skin-related data from a group of users; determining a dataset of skin features from the collected skin-related data; and performing clustering on the dataset of skin features to generate a set of clusters corresponding to a set of skin types.

According to another embodiment of the present invention, the measurement system comprises: a sub-device comprising a color sensor configured to receive an optical signal; and a main device comprising a reception hole that defines a path for the optical signal incident on the color sensor, and a slot into which a film-on which sebum from the user's skin has been collected—is inserted, wherein the slot extends to the reception hole.

In another embodiment of the present invention, the measurement system comprises: a sub-device comprising a protrusion that contacts the skin and an elasticity sensor configured to measure the force by which the skin pushes the protrusion or a displacement of the protrusion; and a main device comprising an electrical connection to the sub-device, configured to acquire data on skin elasticity and to display information regarding the user's skin condition.

### Effects of the Invention

According to the embodiments, it is possible to provide objective information on skin condition and skin type, taking into account the age group to which the subject belongs.

In addition, according to the embodiments, it is possible to quickly and easily acquire information necessary for assessing the skin condition.

Furthermore, according to the embodiments, it is possible to train a machine learning model for assessing skin condition.

The effects of the embodiments are not limited to those described above, and additional effects not mentioned can be clearly understood by those skilled in the art from the present specification and the accompanying drawings.

### BRIEF DESCRIPTION OF THE DRAWINGS

FIG. 1 is a block diagram of a skin type determining device according to an embodiment of the present invention.

FIG. 2 is a graph illustrating the results for subjects after normalizing color tone data and elasticity data using processors according to an embodiment of the present invention.

FIG. 3 is a graph showing normalized oil and moisture data per subject using processors according to an embodiment of the present invention.

FIG. 4 is a graph showing the distribution of subjects after determining a first evaluation index and a second evaluation index using processors according to an embodiment of the present invention.

FIG. 5A shows an example of detecting the reversal point in the color tone index, FIG. 5B shows an example of detecting the reversal point in the elasticity index, FIG. 5C shows an example of detecting the reversal point in the oil index, and FIG. 5D shows an example of detecting the reversal point in the moisture index.

FIG. 6A clarifies the graph of FIG. 5A by indicating the age and group-specific proportions; FIG. 6B clarifies the graph of FIG. 5B by indicating the age and group-specific proportions; FIG. 6C clarifies the graph of FIG. 5C by indicating the age and group-specific proportions; and FIG. 6D clarifies the graph of FIG. 5D by indicating the age and group-specific proportions.

FIG. 7A shows the distribution of color tone values, FIG. 7B shows the distribution of elasticity values, FIG. 7C shows the distribution of moisture values, and FIG. 7D shows the distribution of oil values for the subjects.

FIG. 8A is for the “young” group (ages 10-34), FIG. 8B is for the “aging1” group (ages 35-50), and FIG. 8C is for the “old” group (ages 51-71).

FIG. 9 shows the distribution positions of subjects without applying the first and second age weightings, marked by age group on the same graph format as FIG. 4.

FIG. 10 is a flowchart of a skin type determination method according to an embodiment of the present invention.

FIG. 11A illustrates a set of radial plots exemplifying clusters of skin types for users in a younger age group; FIG. 11B for a middle-aged group; and FIG. 11C for an older age group.

FIG. 12A shows absolute skin scores and phenotypes displayed on a display device;

FIG. 12B visualizes the data for comparison with other skin categories.

FIG. 13 illustrates an example of skin type classification performed by clustering skin-related data of users across all age groups.

FIGS. 14A, 14B, 14C, 14D, 14E, and 14F are examples comparing the classification of skin types obtained by performing clustering by age group with the classification described in FIG. 10.

FIG. 15 is an exemplary process generating clusters corresponding to sets of skin types, which may be used in various embodiments.

FIG. 16 is an exemplary process for determining a user's skin type and providing information for skin improvement, which may be used in various embodiments.

FIG. 17 illustrates an overview of a system according to an embodiment of the present invention.

FIG. 18A is a block diagram of one embodiment of the sub-device shown in FIG. 17, and FIG. 18B is a block diagram of another embodiment of the sub-device shown in FIG. 17.

FIG. 19 is a perspective view of a skin measurement system according to an embodiment of the present invention.

FIG. 20 is an exploded perspective view of the skin measurement system of FIG. 19.

FIG. 21 shows a rear view of the perspective view of FIG. 20.

FIG. 22A is a side view of a first sub-device according to an embodiment of the present invention, and FIG. 22B is a bottom view of the first sub-device according to an embodiment of the present invention.

FIG. 23 illustrates a user holding a handle portion and pressing a button; FIG. 24A illustrates inserting a film into a container; FIG. 24B illustrates staining the film with a staining reagent; FIG. 24C illustrates washing off the staining reagent using a washing buffer; and FIG. 24D illustrates the film after staining has been completed.

FIG. 25 illustrates the usage process of FIG. 24.

FIG. 26A illustrates a color sensor acquiring an optical signal passing through a receiving hole, and FIG. 26B illustrates the first sub-device penetrating the receiving hole within the main device.

FIG. 27 illustrates separating the first sub-device from the main device to measure the skin tone.

FIG. 28A is a side view of a second sub-device according to an embodiment of the present invention, and FIG. 28B is a bottom view of the second sub-device according to an embodiment of the present invention.

FIG. 29 illustrates the second sub-device measuring skin elasticity according to an embodiment of the present invention.

FIG. 30 illustrates front views of the first cap and the second cap according to an embodiment of the present invention.

FIG. 31 illustrates exemplary components of a computing device that may be used in various embodiments.

FIG. 32 is a diagram illustrating the transmission of data as provided in accordance with various embodiments. FIG. 33 is a flowchart illustrating a method for deriving phenotypes by age group.

### BEST MODE FOR CARRYING OUT THE INVENTION

Advantages and features of the present invention, and methods of achieving the same, will become apparent with reference to the embodiments described in detail below in conjunction with the accompanying drawings.

However, the present invention is not limited to the embodiments disclosed below and may be implemented in various different forms. The embodiments are provided merely to ensure complete disclosure of the invention and to fully convey the scope of the invention to those skilled in the art to which the present invention pertains. The invention is defined only by the scope of the claims. Throughout the specification, like reference numerals refer to like elements.

Hereinafter, the present invention will be described with reference to the drawings.

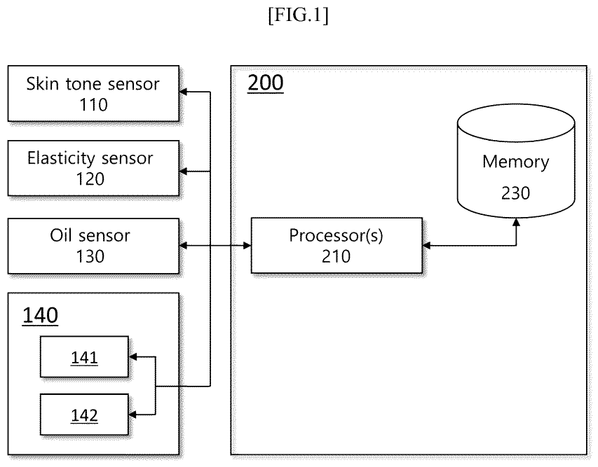

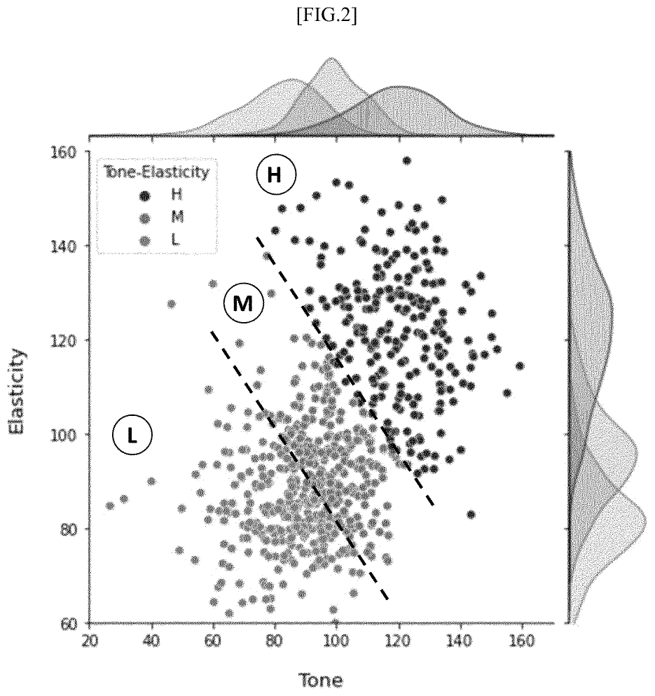

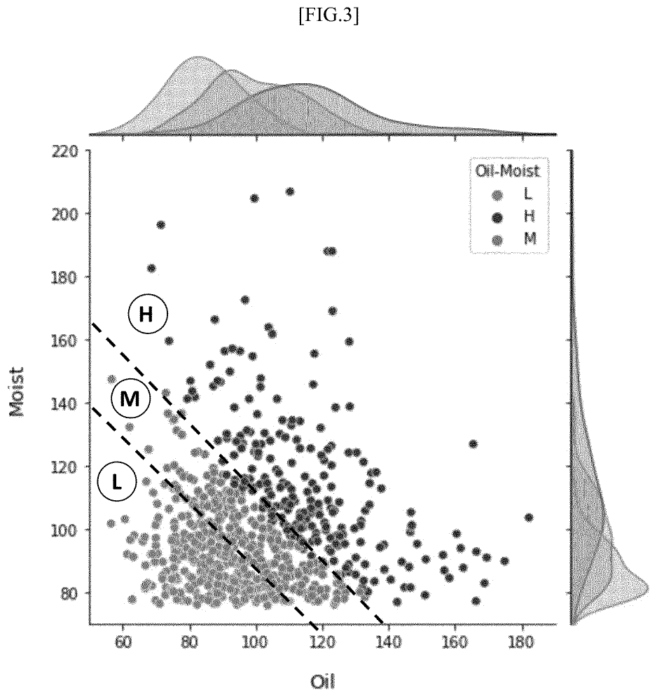

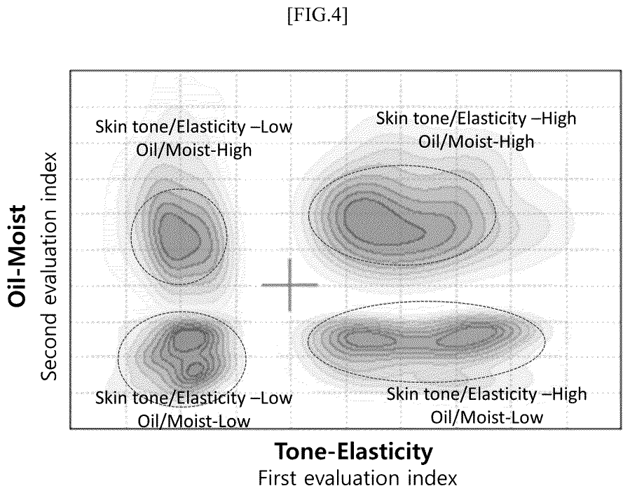

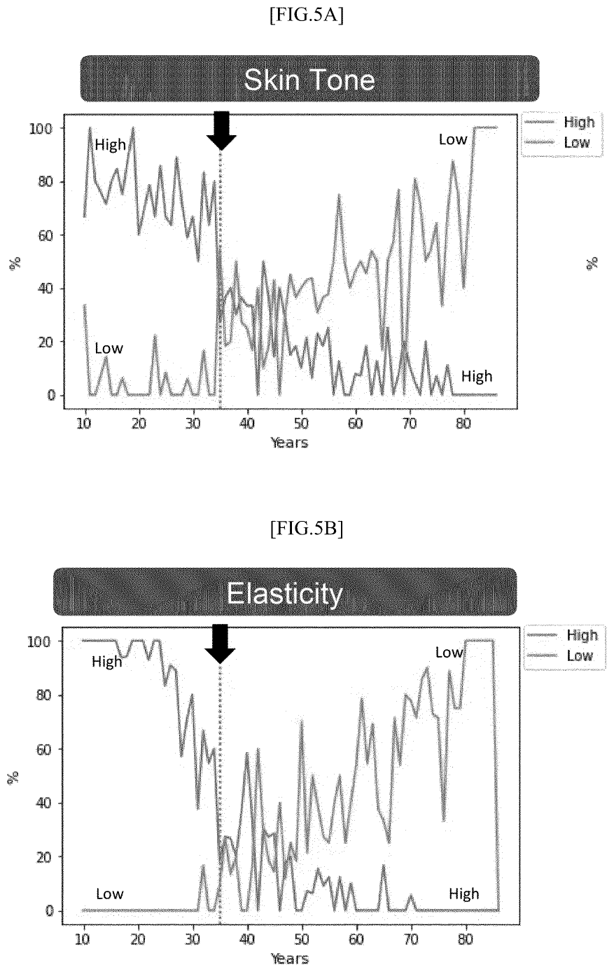

FIG. 1 is a block diagram of a skin type determination apparatus according to various embodiments of the present invention. FIG. 2 is a graph showing the results of test subjects after normalization of tone data and elasticity data by processors (210) according to various embodiments of the present invention. FIG. 3 is a graph showing individual results of test subjects after normalization of sebum data and moisture data by processors (210) according to various embodiments of the present invention. FIG. 4 is a graph showing the distribution of test subjects after determining a first evaluation index and a second evaluation index by processors (210) according to various embodiments of the present invention.

Referring to FIGS. 1 through 4, a skin type determination apparatus or system according to various embodiments of the present invention includes: a skin tone sensor (110) configured to measure the skin tone of a subject; an elasticity sensor (120) configured to measure the elasticity of the subject's skin; a memory (230) configured to store tone data measured by the skin tone sensor (110) and elasticity data measured by the elasticity sensor (120); and one or more processors (210). The one or more processors (210) are configured to normalize the tone data to determine a tone value; normalize the elasticity data to determine an elasticity value; and determine a first evaluation index that classifies the subject's skin type based on the tone value and the elasticity value.

The skin tone sensor (110) may be a color sensor or an optical sensor. The skin tone sensor (110) detects the color of the skin surface. The skin tone sensor (110) receives color information from the skin.

The elasticity sensor (120) detects the elasticity of the skin. The elasticity sensor (120) detects the amount of skin contraction, recovery, and recovery time. The elasticity sensor (120) may be a distance measuring sensor using laser, ultrasound, or infrared. The elasticity sensor (120) receives information on skin contraction and recovery. The recovery time may be determined by the processor (210).

The memory (230) may optionally include one or more non-transitory computer-readable storage media, and may further optionally include high-speed random access memory. Additionally, the memory may optionally include non-volatile memory such as one or more magnetic disk storage devices, flash memory devices, or other non-volatile solid-state memory devices.

The one or more processors (210) operate or execute various software programs and/or sets of instructions stored in the memory (230) to perform various functions related to the skin type determination apparatus and to process data. The processor (210) normalizes the data. Normalization refers to adjusting the range of data values to between 0 and 1. The processor (210) converts all data values to the same unit or to dimensionless numbers.

A skin type determination apparatus or system according to various embodiments of the present invention includes an oil sensor (130), a moisture sensor (140), an elasticity sensor (120), a skin tone sensor (110), and a main device (200). The main device (200) includes a processor and a memory (230). The various sensors (110, 120, 130, 140) and the main device (200) are connected via wired or wireless communication. The main device (200) may be implemented on a cloud server. All or part of the components may be portable mobile devices.

According to FIG. 2, the color data and elasticity data are distributed differently for each subject, and according to FIG. 3, the oil and moisture data are also distributed differently per subject (H: high, M: medium, L: low). The processor (210) may normalize the skin tone data and elasticity data for each subject and store them in the memory (230).

The processor (210) determines the first evaluation index. The first evaluation index is an integrated indicator of tone and elasticity data. As shown in FIG. 4, the processor (210) determines a single first evaluation index based on the tone and elasticity data.

The processor (210) performs operations on two or more variables. The term “operation” refers to mathematical, logical, or other forms of computation performed using two or more variables. For example, the processor (210) may perform mathematical operations. Mathematical operations may include arithmetic operations (addition, subtraction, multiplication, division), exponential operations, logarithmic operations, differentiation, and integration. The processor (210) may determine the first evaluation index and a second evaluation index (described later) by performing a multiplication operation on the tone and elasticity data.

As another example, the processor (210) may perform comparison operations. The processor (210) evaluates at least one of the following cases: greater than, less than, less than or equal to, and greater than or equal to a predetermined threshold, and returns a corresponding value based on the result. Although multiplication is used below as a preferred example of an operation, other mathematical, logical, or comparison operations may also be applied if they help enhance the correlation of the data used to determine skin type or if they are advantageous for data compression. Therefore, the term “operation” in the present invention is not limited to multiplication.

A skin type determination apparatus or system according to various embodiments of the present invention includes a tone sensor (110) for measuring the tone of a subject's skin; an elasticity sensor (120) for measuring the elasticity of the subject's skin; one or more processors (210); a memory (230); and one or more programs stored in the memory (230) and configured to be executed by the one or more processors (210). The one or more programs include instructions for: converting the tone data measured by the tone sensor (110) into a value within a predetermined range to determine a tone value; converting the elasticity data measured by the elasticity sensor (120) into a value within a predetermined range to determine an elasticity value; and determining a first evaluation index that defines the subject's skin type based on the tone value and elasticity value (with multiplication being one preferred example of the operation).

The predetermined range of values may be between 0 and 1. As a specific example, the tone data measured by the tone sensor (110) may be converted into values ranging from 0 to 1. As another specific example, the elasticity data measured by the elasticity sensor (120) may be converted into values ranging from 0 to 1. The processor (210) adjusts the data columns to fall within the range of 0 to 1. The processor (210) may also perform machine learning.

The processor (210) may perform data standardization. Standardization may be conducted under the assumption that the data follows a normal (bell-shaped) distribution. The processor (210) may transform the data so that the mean becomes 0 and the standard deviation becomes 1.

Through normalization or standardization, the processor (210) can facilitate data learning. Instead of focusing on a particular column, the processor (210) learns all columns equally. After performing both normalization and standardization followed by machine learning, the processor (210) may compare the results and determine whether to standardize or normalize the data.

A skin type determination apparatus or system according to various embodiments of the present invention includes a sebum sensor (130) for measuring the sebum level of a subject's skin; a moisture sensor (140) for measuring the moisture level of the subject's skin; one or more processors (210); a memory (230); and one or more programs stored in the memory (230) and configured to be executed by the one or more processors (210). The one or more programs include instructions for: converting the sebum data measured by the sebum sensor (130) into a value within a predetermined range to determine a sebum value; converting the moisture data measured by the moisture sensor (140) into a value within a predetermined range to determine a moisture value; and determining a second evaluation index that defines the subject's skin type based on the sebum value and the moisture value (with multiplication being one preferred example of the operation).

According to FIG. 2, the sebum and moisture data are distributed differently for each subject (H: high, M: medium, L: low). The processor (210) may normalize the sebum and moisture data for each subject and store the results in the memory (230). The processor (210) may use the sebum and moisture data to determine a single second evaluation index that integrates both.

The processor (210) determines the second evaluation index, which is an index that integrates the sebum and moisture data. As shown in FIG. 4, the processor (210) also determines a first evaluation index based on the tone data and elasticity data (with multiplication being one preferred example of the operation). Using both the first and second evaluation indices, the processor (210) may display the skin condition of the subject on a single plane.

The moisture sensor (140) includes a first moisture measuring device that measures transepidermal water loss (TEWL) of the subject's skin, and a second moisture measuring device that measures hydration (HD) of the subject's skin. The one or more programs convert the transepidermal water loss and hydration values into values within a predetermined range, and determine the moisture value using the following formula:

$$M = \frac{H D}{T E W L}$$

Here, M is the moisture value, HD is the hydration level of the skin, and TEWL is the transepidermal water loss of the skin.

The processor (210) may determine a single moisture value by integrating the hydration level and the transepidermal water loss to generate an index representing the skin's moisture condition. The processor (210) judges that the lower the skin's transepidermal water loss, the better the skin barrier function is maintained. The processor (210) determines that the lower the transepidermal water loss, the higher the skin's moisturizing ability. The processor (210) determines that the higher the skin's hydration level, the better the moisturizing ability of the skin. The processor (210) determines that the higher the value of the moisture index (M), the better the skin's moisturizing ability.

The first moisture measuring device measures the amount of moisture lost from the skin per minute, quantifying the amount of moisture decrease per minute. The skin's transepidermal water loss refers to the moisture loss from the skin barrier located in the outermost layer of the skin, the stratum corneum. The second moisture measuring device measures the amount of moisture contained within the skin.

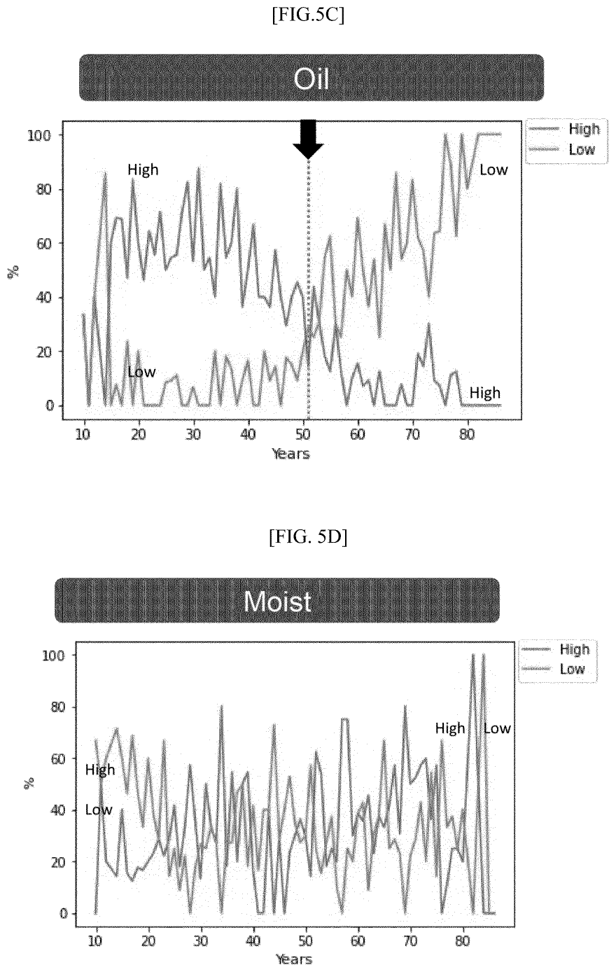

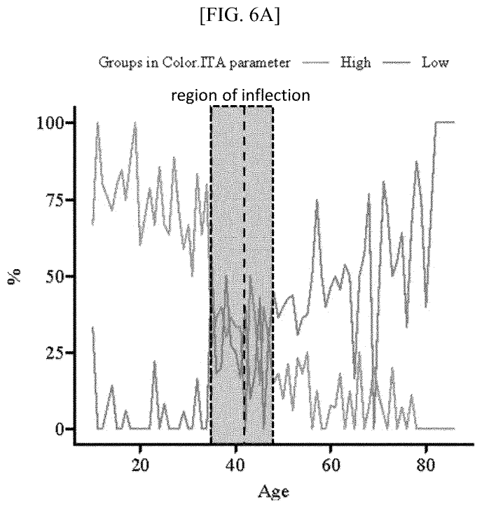

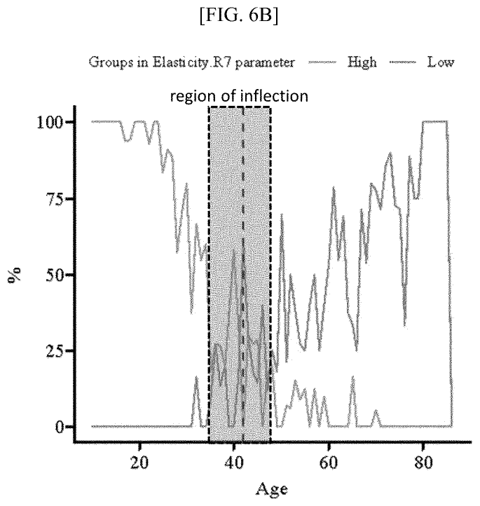

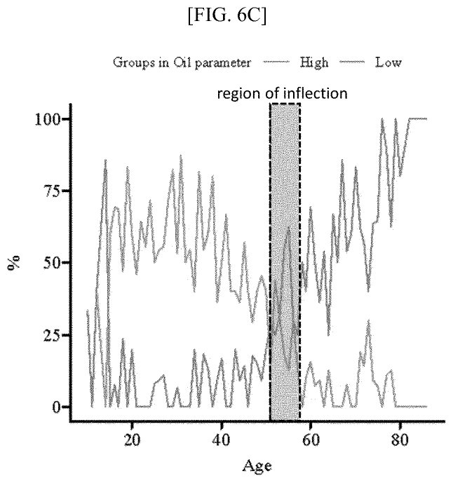

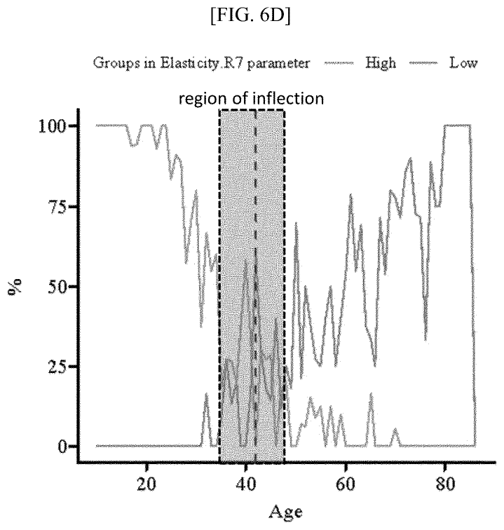

FIG. 5a illustrates an example where the processor(s) (210) according to various embodiments of the present invention identify the point at which an inversion phenomenon occurs in the tone index. FIG. 5b illustrates an example where the processor(s) (210) identify the point at which an inversion phenomenon occurs in the elasticity index. FIG. 5c illustrates an example where the processor(s) (210) identify the point at which an inversion phenomenon occurs in the oil index. FIG. 5d illustrates an example where the processor(s) (210) identify the point at which an inversion phenomenon occurs in the moisture value. FIGS. 6a, 6b, 6c, and 6d clarify the charts of FIGS. 5a, 5b, 5c, and 5d, respectively, by indicating the age and the proportion of each group.

The skin type determination apparatus according to various embodiments of the present invention includes a sebum sensor (130) for measuring the subject's skin sebum; and a moisture sensor (140) for measuring the subject's skin moisture.

One or more programs include instructions for quantifying individual skin condition data measured by the sensors according to predetermined criteria to classify groups, selecting data types whose group proportion change patterns by age are similar, and determining the subject's skin condition based on these.

The pattern may be determined based on the similarity of age groups where an inversion phenomenon occurs in the ranking of group proportions. Data types with similar group proportion change patterns may be tone data and elasticity data.

The meaning of the inversion phenomenon is explained.

According to FIGS. 5a and 6a, in the case of the subjects' tone values, the proportions of the high group and the low group change around age 34, and the pattern is completely inverted by the late 40s. That is, under the age of 34, the high group is the majority, but after age 34, the low group gradually becomes the majority.

Again, according to FIGS. 5b and 6b, in the case of the subjects' elasticity values, the proportions of the high group and the low group change around age 34, and the pattern is completely inverted by the late 40s. That is, under the age of 34, the high group is the majority, but after age 34, the low group gradually becomes the majority.

As such, both tone values and elasticity values exhibit the inversion phenomenon, and the age at which it occurs is the same. Therefore, the data for tone values and elasticity values have the same pattern. Tone values and elasticity values are highly correlated data.

Therefore, when the tone values and elasticity values are multiplied, the characteristics are amplified, allowing clearer distinction of the data features.

Meanwhile, referring to FIGS. 5c and 6c, the oil values exhibit an inversion phenomenon, but the age range in which this occurs is clearly distinct from that of the tone and elasticity values. Referring to FIGS. 5d and 6d, the moisture values do not exhibit an inversion phenomenon. Although moisture values represent the individual's skin condition, they have a low correlation with age.

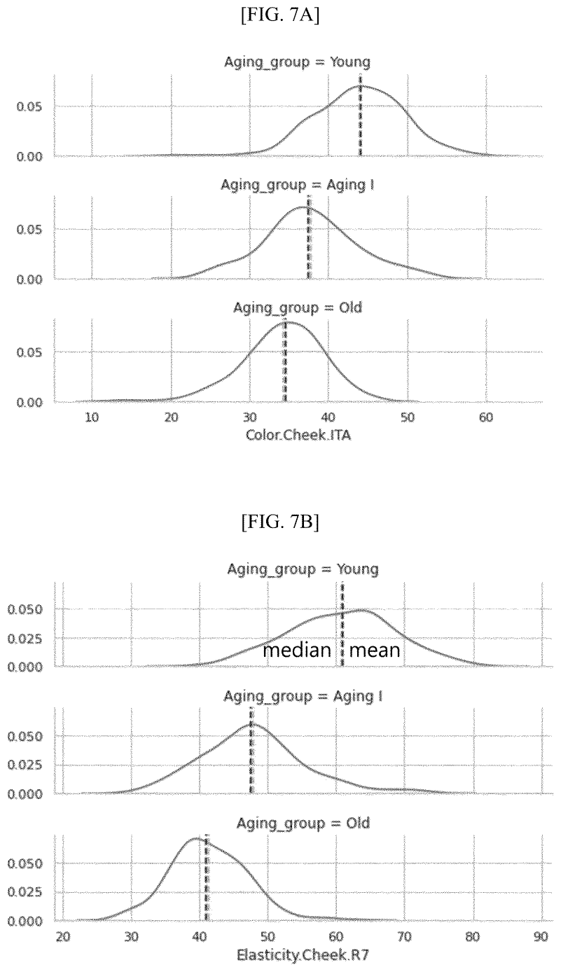

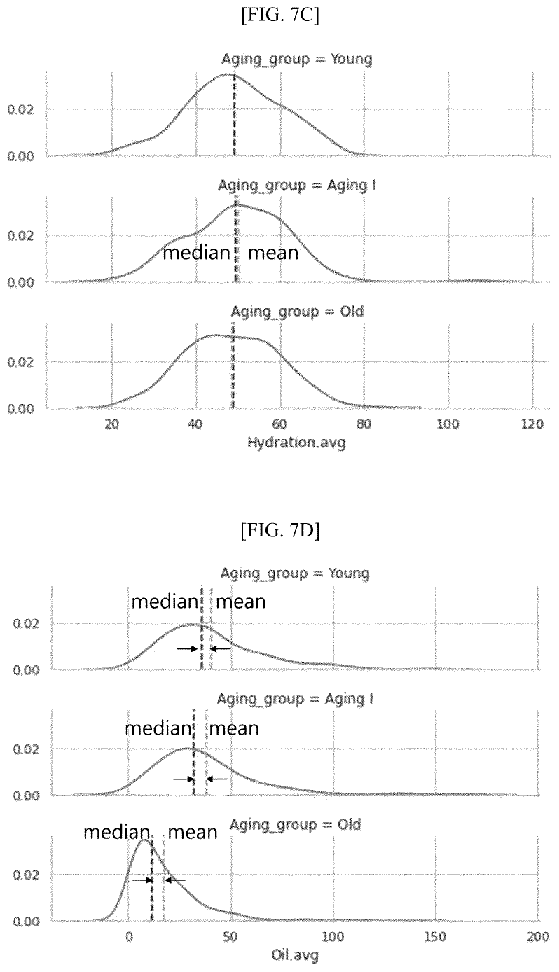

FIG. 7a expresses the subjects' tone values as a normal distribution curve. FIG. 7b expresses the subjects' elasticity values as a normal distribution curve. FIG. 7c expresses the subjects' moisture values as a normal distribution curve. FIG. 7d expresses the subjects' oil values as a normal distribution curve.

One or more programs according to various embodiments of the present invention include commands to select the remaining data whose group proportion change patterns are dissimilar and determine the subject's skin condition. The remaining data whose group proportion change patterns are dissimilar are the oil data measured by the oil sensor (130) and the moisture data measured by the moisture sensor (140).

Referring to FIGS. 7a, 7b, and 7c, the median and mean values coincide. According to FIG. 7d, the oil values show a discrepancy between the mean and median values. According to FIG. 7d, the oil values have an asymmetrical or long-tailed distribution, where outliers or unusual values affect the mean value.

There exist abnormally high oil value individuals (in the tail section), and it is desirable to reduce the influence of these outliers.

According to various embodiments of the present invention, multiplying the oil value (oil data) by the moisture value (moisture data) can reduce the influence of outliers present in the oil data and enhance the pattern of normal data. Therefore, data quality and judgment accuracy are improved. In addition, the multiplication of the oil value (oil data) by the moisture value (moisture data) can be used as an indicator to determine individual skin conditions that are independent of age.

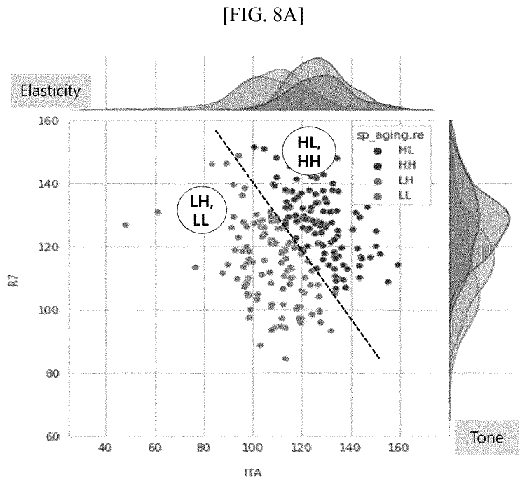

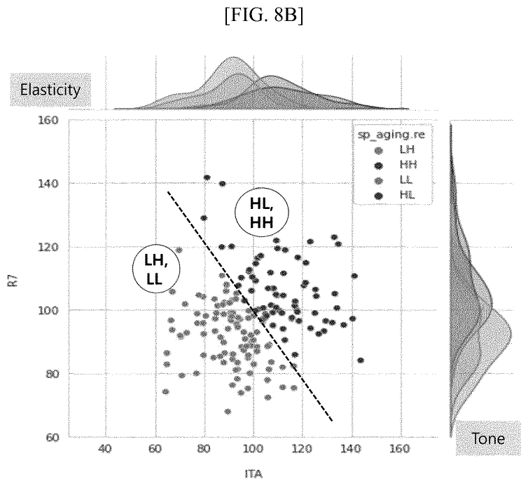

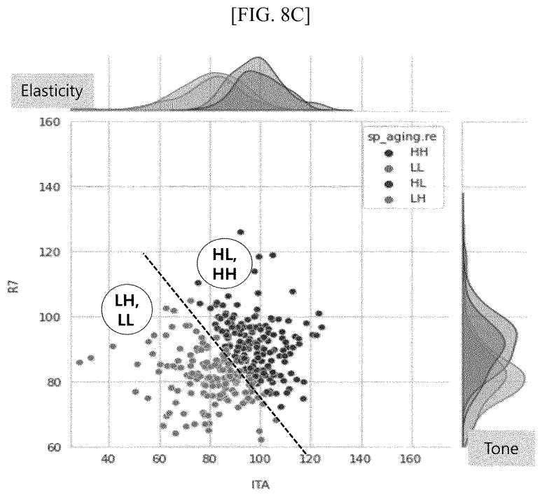

FIG. 8a is a scatter plot showing the High group and Low group distinguished by values obtained by normalizing and then multiplying the tone data and elasticity data of the young group (ages 10 to 34). FIG. 8b is a scatter plot showing the High group and Low group distinguished by values obtained by normalizing and then multiplying the tone data and elasticity data of the aging1 group (ages 35 to 50). FIG. 8c is a scatter plot showing the High group and Low group distinguished by values obtained by normalizing and then multiplying the tone data and elasticity data of the old group (ages 51 to 71).

Referring to FIGS. 8a, 8b, and 8c, if the product of the X-axis value (tone value) and the Y-axis value (elasticity value) of any point exceeds a certain threshold, it belongs to the High group; if it is below, it belongs to the Low group.

FIG. 8a analyzes a relatively young subject group, where the boundary line (dotted line) separating the High and Low groups is located at the upper right. As in FIGS. 8b and 8c, the boundary line shifts toward the lower left as the age group increases. Commonly, subject results are concentrated near the boundary line. This shows that the multiplication result of the tone and elasticity values has few outliers, making it suitable as a criterion for determining skin condition.

Meanwhile, since skin condition is absolutely influenced by age, an absolute skin condition assessment ignoring age is meaningless. The shifting boundary line according to age demonstrates that it serves as a criterion for relative skin condition assessment considering age. Therefore, the multiplication result of tone and elasticity values becomes a criterion for relative skin condition assessment considering age.

According to various embodiments of the present invention, the condition of subjects can be assessed considering age by classifying them into four phenotypes.

The processor (210) can use the multiplication results of the tone data and elasticity data, and the multiplication results of the oil data and moisture data, to classify the subject's skin condition into four groups: HH, HL, LH, and LL. The processor (210) can distinguish the four groups as follows: HH (both toneelasticity and oilmoisture are high), HL (toneelasticity is high but oilmoisture is low), LL (both toneelasticity and oilmoisture are low), and LH (toneelasticity is low but oilmoisture is high). These four groups exhibit characteristic distributions on the graph.

The skin type determination device according to various embodiments of the present invention can distinguish the subject's skin type by age group. That is, it can assess relative skin condition by age group, rather than absolute skin condition. Since skin condition is absolutely influenced by age, assessments made without considering age may be meaningless.

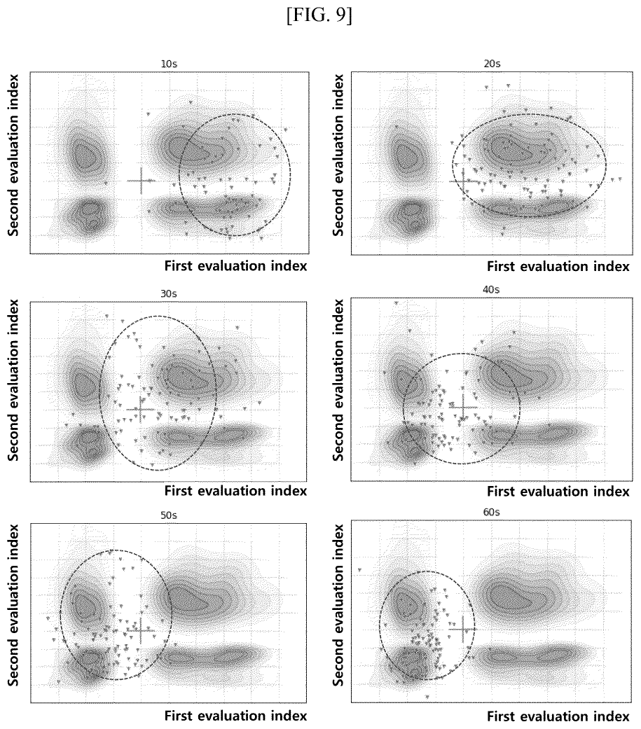

FIG. 9 shows the distribution positions of individual subjects by age group plotted on the graph of FIG. 4, without applying the first and second age weighting factors.

The first evaluation index is the multiplication result of tone value and elasticity value, and the second evaluation index is the multiplication result of oil value and moisture value.

Referring to FIG. 9, it can be seen that subject groups in their teens, twenties, thirties, forties, fifties, and sixties (represented by inverted triangle shapes) form clusters that gradually shift. This indicates that the first and second evaluation indices intuitively show relative results of the subject's skin condition by comparing with groups of the same age.

According to various embodiments of the present invention, even if a subject is classified as LL based on the YOUNG group standard, the same subject may be classified as HH or HL based on the OLD group standard, allowing for appropriate skin treatment recommendations.

The centers, regions, or boundaries of the phenotypic clusters shift according to age groups. The processor (210) can determine the features of these centers or clusters and classify the relative skin condition of individual subjects accordingly.

One or more programs according to various embodiments of the present invention determine the first phenotype by performing an operation (preferably multiplication) of the first evaluation index with a first age weighting factor determined based on the subject's age.

One or more processors (210) are configured to determine the second phenotype by performing an operation (preferably multiplication) of the second evaluation index with a second age weighting factor determined based on the subject's age.

For example, the processor (210) may classify subjects into three stages: Young group for those 34 years old and under, Aging I group for those aged 35 to 50, and Old group for those 51 and older. The first and second age weighting factors may also vary depending on gender. The processor (210) can apply different first and second age weighting factors for each age group.

The processor (210) may determine the first and second age weighting factors differently according to age by machine learning. The first and second age weighting factors may also be predetermined. The first and second age weighting factors can be stored in memory (230).

The processor (210) can determine the age at which a reversal phenomenon occurs by comparing the tone indices of many collected subjects. For example, the processor (210) may determine that subjects under 35 mostly have tone indices exceeding 0.5, but those aged 35 and above mostly have tone indices below 0.5 (in FIGS. 5a, 5b, 5c, 5d, majority is marked as high and minority as low, and arrows indicate the point of reversal).

In this case, the processor (210) determines that age 35 is the age at which the reversal phenomenon occurs for the tone index. Accordingly, the processor (210) may set the first age weighting factor to 1 for those aged 35 and above, and 0.7 for those under 35. Similarly, for the elasticity index, the processor (210) may also determine age 35 as the reversal point.

The processor (210) can determine the age at which a reversal phenomenon occurs by comparing oil values of many collected subjects. For example, the processor (210) may divide subjects into three groups: under 30, 30 to under 50, and 50 and above, and determine the second age weighting factor accordingly.

For example, the processor (210) may determine that until age 15, most subjects have oil values exceeding 0.5; between ages 15 and under 50, most have oil values below 0.5; and at age 50 and above, most have oil values above 0.5.

In this case, the processor (210) determines ages 15 and 50 as points of reversal and sets the second age weighting factor differently before and after these ages.

The processor (210) considers relative differences due to age by applying the first and second age weighting factors. It determines the subject's skin condition relative to the same age group by applying these age weighting factors.

Referring to FIG. 6, the skin condition of subjects by age group is clustered in certain areas. However, skin condition should be judged relatively within the same age range. Therefore, the processor (210) needs to consider the distribution by age.

For example, if the processor (210) determines that a subject in their teens has objectively better skin condition than a subject in their seventies, but comparatively lower elasticity than other teens, it can assign a mark indicating that the first phenotype is low, insufficient, or poor relative to the same age group. Conversely, the processor (210) may assign a mark indicating that the first phenotype is high, sufficient, or excellent relative to the same age group for a subject in their seventies.

The processor (210) may determine the second age weighting factor and the second phenotype in the same manner as the first age weighting factor and the first phenotype.

According to embodiments, the memory (230) may store information about the first age weighting factor and the second age weighting factor in advance.

In a skin type determination device according to various embodiments of the present invention, including a tone sensor (110), elasticity sensor (120), moisture sensor (140), oil sensor (130), and one or more processors (210), the method includes: measuring tone, elasticity, oil, and moisture of the subject's skin; and determining a first evaluation index and a second evaluation index by one or more processors (210). The first evaluation index is determined by the processor (210) converting tone data into a value within a predetermined range to determine the tone value, converting elasticity data into a value within a predetermined range to determine the elasticity value, and calculating (one preferred example is multiplication) the tone value and elasticity value. The second evaluation index is determined by the processor (210) converting oil data into a value within a predetermined range to determine the oil value, converting moisture data into a value within a predetermined range to determine the moisture value, and calculating (one preferred example is multiplication) the oil value and moisture value.

The skin type determination device or system according to various embodiments of the present invention includes an oil sensor (130) measuring oil of the subject's skin; a moisture sensor (140) measuring moisture of the subject's skin; a memory (230) storing oil data measured by the oil sensor (130) and moisture data measured by the moisture sensor (140); and one or more processors (210). The one or more processors (210) are configured to normalize the oil data to determine the oil value; normalize the moisture data to determine the moisture value; and based on the oil value and moisture value (one preferred example is multiplication), determine a second evaluation index that defines the subject's skin type.

The skin type determination device or system according to various embodiments of the present invention includes a tone sensor (110) measuring tone of the subject's skin; an elasticity sensor (120) measuring elasticity of the subject's skin; one or more processors (210); memory (230); and one or more programs stored in the memory (230) and configured to be executed by the one or more processors (210). The one or more programs include instructions to convert tone data measured by the tone sensor (110) into a value within a predetermined range to determine the tone value, convert elasticity data measured by the elasticity sensor (120) into a value within a predetermined range to determine the elasticity value; and calculate (one preferred example is multiplication) the tone value and elasticity value to determine a first evaluation index defining the subject's skin type.

The value within the predetermined range may be between 0 and 1.

The one or more programs determine a first phenotype by calculating (one preferred example is multiplication) the predetermined first age weighting factor based on the subject's age with the first evaluation index, and store the first phenotype and second phenotype in the memory (230).

The skin type determination device or system according to various embodiments of the present invention includes a moisture sensor (140) measuring moisture of the subject's skin; one or more processors (210); memory (230); and one or more programs stored in the memory (230) and configured to be executed by the one or more processors (210). The one or more programs convert moisture data measured by the moisture sensor (140) into a value within a predetermined range to determine a moisture value. The moisture sensor (140) includes a first moisture measuring device that measures moisture loss of the subject's skin and a second moisture measuring device that measures the hydration content of the subject's skin. The one or more programs convert moisture loss and hydration content into values within a predetermined range and include instructions to determine the moisture value and assess the skin condition using the following formula.

$$M = \frac{H D}{T E W L}$$

Here, M is the moisture value, HD is the hydration content, and TEWL is the trans-epidermal water loss (moisture loss).

The skin type determination device or system according to various embodiments of the present invention includes a color sensor (110) that measures the subject's skin tone; an elasticity sensor (120) that measures the skin elasticity; an oil sensor (130) that measures the skin oil content; a moisture sensor (140) that measures the skin moisture; a memory (230) that stores color data measured by the color sensor (110) and elasticity data measured by the elasticity sensor (120); and one or more processors (210).

The one or more processors (210) are configured to convert the color data into a value within a predetermined range to determine a color value; convert the elasticity data into a value within a predetermined range to determine an elasticity value; multiply the color value and elasticity value to determine a first evaluation index defining the subject's skin type; convert the oil data into a value within a predetermined range to determine an oil value; convert the moisture data into a value within a predetermined range to determine a moisture value; and based on the oil value and moisture value (preferably by multiplication), determine a second evaluation index defining the subject's skin type.

The memory (230) stores one or more standardization or normalization programs executed by the one or more processors (210) to convert data into values within the predetermined range.

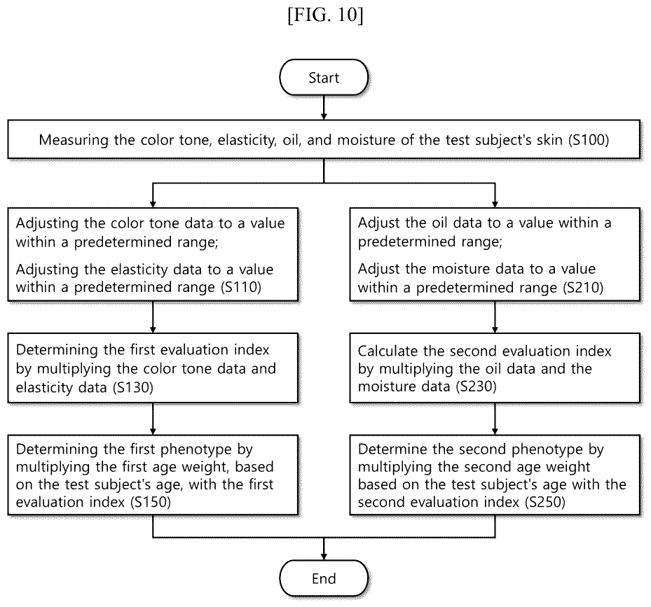

FIG. 10 is a flowchart of the skin type determination method according to various embodiments of the present invention.

Referring to FIG. 10, the skin type determination method according to various embodiments of the present invention, using a skin type determination device comprising a color sensor (110), elasticity sensor (120), moisture sensor (140), oil sensor (130), and one or more processors (210), includes the steps of: measuring the subject's skin tone, elasticity, oil content, and moisture (S100); and determining a first evaluation index and a second evaluation index by the one or more processors (210), wherein the first evaluation index is determined by converting color data into a value within a predetermined range to determine a color value, converting elasticity data into a value within a predetermined range to determine an elasticity value (S110), and calculating the color value and elasticity value (preferably by multiplication) (S130); and the second evaluation index is determined by converting oil data into a value within a predetermined range to determine an oil value, converting moisture data into a value within a predetermined range to determine a moisture value (S210), and calculating the oil value and moisture value (preferably by multiplication) (S230).

According to various embodiments of the present invention, the processor (210) performs a step (S150) of determining a first phenotype by multiplying a first age weight, determined based on the subject's age, with the first evaluation index. According to various embodiments of the present invention, the processor (210) performs a step (S250) of determining a second phenotype by multiplying a second age weight, determined based on the subject's age, with the second evaluation index. One or more processors (210) may convert the moisture loss amount and hydration amount into values within a predetermined range, and determine the moisture value using these. The formula for determining the moisture value has been described above.

The skin type determination device and/or system according to various embodiments of the present invention may denote the first phenotype and the second phenotype by appending an age notation as shown below, instead of multiplying by the age weight.

$$Skin type \{ \begin{matrix}H ( \begin{matrix}H \\ L\end{matrix} ) \\ L ( \begin{matrix}H \\ L\end{matrix} )\end{matrix} \\ ––— \text{—––} \\ {1}^{st} phenotype {2}^{nd} phenotype$$

The memory (230) may store the subject's skin condition classified into phenotypes such as H/H, H/L, L/H, and L/L. In the phenotype, the prefix H or L may indicate whether the skin tone and elasticity are high or low, or excellent or deficient. The suffix H or L in the phenotype may indicate whether the sebum and moisture are high or low, or excellent or deficient. The phenotype is used as data for improving the subject's skin condition. The above-described contents may also be summarized by the following formula.

Referring to FIG. 33, Step A relates to the age group derivation algorithm (Female & 5 parameters, N=775).

The criteria for high and low age may vary, but in the above embodiment, young is defined as 34 years or younger, Aging1 as 35 to 50 years, and Old as 51 years or older.

Thereafter, five parameters—skin tone, elasticity, sebum, moisture, and moisture loss—are measured using sensors. In FIG. 33, Step B distinguishes between high and low based on skin tone and elasticity values (Clinical data & Survey, N=705). In FIG. 33, Step C distinguishes between high and low based on sebum and moisture values.

The formulas for standardizing various data according to the present invention are as follows.

$$SS Function : Standard Scalar ( mean = 100 , std = 20 )$$

$$SS ( x ) = \frac{20 \times ( x - {x}_{mean} )}{{x}_{std}} + 100$$

The above formula is intended to convert data into values within the range of 0 to 200.

$$Tone and Elasticity value ( {TE}_{ss} )$$

$${TE}_{ss} = SS ( Color . {ITA}_{ss} \times Elasticity . R {7}_{ss} ) = SS \{ SS ( Color . Cheek . ITA ) \times SS ( Elasticity . Cheek . R 7 ) \}$$

The above formula represents a multiplication operation of standardized or normalized data related to tone and elasticity.

$$Oil and Moist value ( {OM}_{ss} )$$

$${OM}_{ss} = SS ( {Oil}_{ss} \times {Moist}_{ss} ) = SS \{ SS ( {Oil}_{mean} ) \times SS ( Moist ) \} = SS \{ SS ( {Oil}_{mean} ) \times SS ( \frac{{Hydration}_{mean}}{{TEWL}_{mean}} ) \}$$

The above formula represents a multiplication operation of standardized or normalized data related to oil and moisture.

$${Oil}_{mean} = \frac{Oil . Forehead + Oil . Nose + Oil . Cheek}{3}$$

The above formula represents the formula used to derive oil data. Oil is measured from the forehead, nose, and cheeks, and the average value is used.

$${Hydration}_{m e a n} = \frac{Hydration . Forehead + Hydration . Cheek}{2}$$

The above formula represents the formula used to derive moisture content data. Moisture is measured from the forehead and cheeks, and the average value is used.

$${TEWL}_{mean} = \frac{TEWL . Forehead + TEWL . Cheek}{2}$$

The above formula represents the formula used to derive transepidermal water loss (TEWL) data. Moisture is measured from the forehead and cheeks, and the average value is used.

The method for classifying aging groups is as follows.

$$GRP ( {x}_{ss} ) = \{ \begin{matrix}L , {x}_{ss} \leq Q 1 \\ M , Q 1 < {x}_{ss} \leq Q 3 \\ H , {x}_{ss} < Q 3\end{matrix} \\ {Q}_{1} = Quantile ( {x}_{ss} , 1 ) , {Q}_{3} = Quantile ( {x}_{ss} , 3 )$$

Quantiles are divided into three groups and were used to distinctly separate the middle group (grey zone) from the upper group (H) and the lower group (L).

Regarding the above formula, first, for the SS (x) of tone, elasticity, oil, and moisture, the GRP(xss) is executed.

$$GRP ( Color . {ITA}_{s s} ) , GRP ( Elasticity . R {7}_{s s} ) , GRP ( {Oil}_{ss} ) , GRP ( {Moist}_{ss} ) .$$

Next, age is extracted.

From the clinical measurement values, the percentage (%) for GRP (x) by age is checked. Then, the age at which the sample ratio of H type becomes lower than that of L type is identified.

The derived example is as follows.

$${Age}_{ITA} = 35 , {Age}_{R 7} = 36 , {Age}_{Oil} = 51 \\ AgingGroup = \{ \begin{matrix}Young , 10 \leq Age \leq 34 \\ Aging l , 35 < Age \leq 50 \\ Old , 51 \leq Age\end{matrix}$$

The following formula indicates that a value is expressed as “high” if it exceeds a predefined threshold, and as “low” if it is below that threshold.

$$SP ( x ) = \{ \begin{matrix}L , x < {x}_{mean} \\ H , x > {x}_{mean}\end{matrix}$$

The following describes how the tone-elasticity (TE) related phenotype can be expressed as either H or L, as shown above.

$$T {E}_{g r o u p} = S P ( T {E}_{ss} )$$

The following describes how the oil-moisture (OM) related phenotype can be expressed as either H or L.

$${OM}_{group} = \{ \begin{matrix}SP ( {OM}_{ss} ) , & {TE}_{group} = H \\ SP ( {OM}_{ss} ) , & {TE}_{group} = L\end{matrix}$$

The following lists the age group together with the tone-elasticity (TE) related phenotype. In the example below, the subject belongs to the Young group.

$$Young . {TE}_{group} = {TE}_{g r o u p} ( {TE}_{s s} in Young samples )$$

The following lists the age group together with the oil-moisture (OM) related phenotype. In the example below, the subject belongs to the Young group.

$$Young . {OM}_{group} = \{ \begin{matrix}SP ( {OM}_{ss} in Young samples ) , & Young . {TE}_{group} = H \\ SP ( {OM}_{ss} in Young samples ) , & Young . {TE}_{group} = L\end{matrix}$$

Information on tone-elasticity (TE), oil-moisture (OM), and age can be represented as follows.

$$Young . KSC = Young . {TE}_{g r o u p} + Young . {OM}_{group}$$

$$KSC \{ \begin{matrix}H ( \begin{matrix}H \\ L\end{matrix} ) \\ L ( \begin{matrix}H \\ L\end{matrix} )\end{matrix} \\ —– — – \\ TE type OM type$$

The effects of the present invention are described as follows.

According to the present invention, the processor (210) can obtain a first evaluation index and a second evaluation index by multiplying four types of data.

Additionally, according to the present invention, the processor (210) can group subjects by determining the first and second evaluation indices, and use the normalized or standardized data for machine learning.

Furthermore, the processor (210) of the present invention enables relative evaluation considering age by applying a first age weighting and a second age weighting to tone, elasticity, oil, and moisture.

In addition, the processor (210) of the present invention can objectively evaluate skin condition by processing only four types of data. Elasticity is highly associated with pores and wrinkles. Therefore, the first and second phenotypes described below can be considered as effectively including all six indicators, including pores and wrinkles.

Moreover, according to the present invention, the processor (210) can independently assess the skin's moisture condition without considering age, by using the moisture value.

In many embodiments, a wearable computer or device may include a touch-sensitive display through which a user can enter information such as their age, skin condition, lifestyle, or answers to displayed questions.

In such examples, the wearable device may wirelessly communicate (e.g., via Bluetooth® or Wi-Fi) with at least one other computing device associated with the user.

Each of these devices may include some type of presentation mechanism, such as a display screen, that can be used to convey information to the user of the device.

A wearable device or a device with an embedded sensor for measuring skin condition may communicate with other user devices such as smartphones, desktop computers, or laptops.

The reason for building a system by combining multiple devices in this way is that, considering usability, battery life, size, data analysis, and presentation from various perspectives, it may be more efficient to transmit data to another device.

As discussed in more detail later in the present invention, data about the user can be collected using a measuring device that includes at least one sensor. The measuring device may comprise a sub-device (310), a main device (320), or a system including at least one sub-device (310) and a main device (320).

The sub-device (310) and/or main device (320) may include various sensors such as a color sensor (311, 411), an elasticity sensor (312, 423), a sebum sensor (313), and a moisture sensor (314, 427), which can be used to measure or detect information about the user. At least some of the collected data may be analyzed for the purpose of determining the user's corresponding skin type.

In at least one embodiment, the color sensor (423) can be utilized as a sebum sensor (313).

The user interface may provide the user with the ability to input specified data.

At least one of the main device (320) or the sub-device (310) may be a wearable device. The main device (320) may provide users with recommendations or information about customized cosmetics based on their skin type.

In at least one embodiment, skin-related data may be used to analyze data from multiple users and generate a set of skin types to which users may belong.

In at least one embodiment, skin-related data collected for a user may be analyzed and used to determine the skin type that the user belongs to.

The skin type may be represented as a phenotype and can be presented in a manner understandable to the user, such as through graphs or comparative images.

For example, the “yHH” skin phenotype refers to a case where the user is relatively young (“young”), the first phenotype has a high rating (“High”), and the second phenotype also has a high rating (“High”).

As another example, the “OHL” skin phenotype refers to a case where the user is older (“old”), the first phenotype has a high rating, and the second phenotype has a low rating (“Low”).

As yet another example, the “aLL” skin phenotype refers to a case where the user is in a middle age range, the first phenotype has a low rating, and the second phenotype also has a low rating (“Low”).

In at least one embodiment, the ratings of the first and second phenotypes may vary according to weights applied based on age. Various other labels, types, and characteristics may also be used within the scope of different embodiments.

In at least some embodiments, the skin phenotype displayed on the main device (320) may also be provided to a related device such as a smartphone. The user may also obtain information through a browser on a desktop, smart device (340), or laptop computer accessible via a user account.

In various embodiments of the present invention, a skin type identification device includes: a display device (323); a measurement system that obtains elasticity data, tone data, moisture data, and sebum data of the skin; at least one processor (321); and a memory (327) storing instructions. When executed by the at least one processor (321), the instructions: acquire the user's skin-related data using the measurement system; determine user values for a set of skin metrics from the skin-related data; compare the user values with skin metric values of each of a set of skin types to identify the skin type that most closely represents the user's skin; display the information about the identified skin type for the user on the display device (323); and determine the skin type using a first evaluation index derived from multiplying tone data and elasticity data, and a second evaluation index derived from multiplying sebum data and moisture data.

In various embodiments of the present invention, the skin type identification device includes: one or more processors (321); and a memory (327) comprising instructions. When executed by the one or more processors (321), the instructions: acquire skin-related data for a user collected at least once from one or more electronic devices; determine user values for a set of skin metrics from the skin-related data; compare the user values with skin metric values of each of a set of skin types to identify the skin type that most closely represents the user's skin; and generate at least one recommendation to be presented to the user to assist in improving the user's skin, wherein the at least one recommendation is determined at least in part based on the user values for the skin metrics and the skin type identified for the user.

In various embodiments of the present invention, four typical skin types observed across identified groups have been defined and developed. In such examples, six skin metrics selected or defined from the data can be used to evaluate a user's skin type.

Next, a user profile may be generated at least partially based on each skin type and the values of these advanced skin metrics, allowing the user to understand not only their skin type but also the evaluation results using their skin phenotype.

In at least one embodiment, the skin profile may help quantify the skin condition as “good,” “poor,” “better,” or “worse” compared to one or more previous periods (assuming multiple measurements) or compared to other users, and this information may be used to create a skin report card.

To determine the user's skin type, a determination of possible skin types is first made. This may include determining skin types based on data collection and analysis for a large number of users. This may involve analyzing any potential skin-related data collected, detected, or otherwise provided by numerous arbitrary sources.

In at least some embodiments, the skin-related data may be aggregated for analysis. This data may be analyzed using various approaches to select the data to be included in the analysis.

Then, the selected data may be further analyzed in an attempt to reduce the dimensionality of the dataset. Subsequently, an effort may be made to reduce the dataset to a relatively small number of factors that particularly represent different skin types.

In at least some embodiments of the present invention, it may be determined that wrinkle data and pore data are highly related to skin elasticity data. In this case, the dataset may be constructed using only the remaining factors, excluding wrinkle and pore data.

In at least one embodiment, this may include performing clustering using one or more clustering algorithms such as k-means clustering with different clustering parameters.

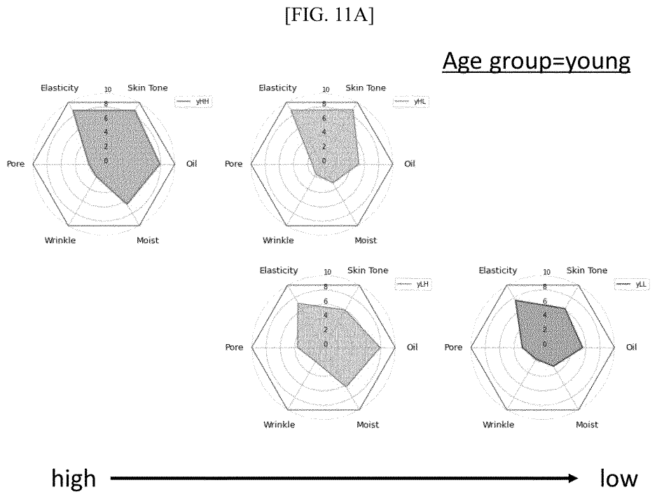

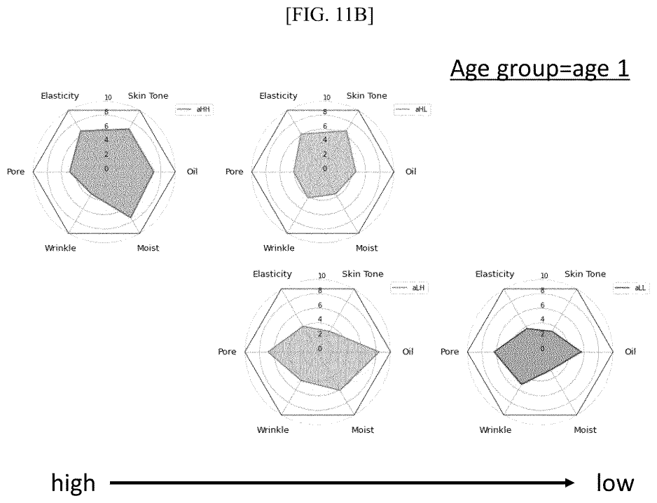

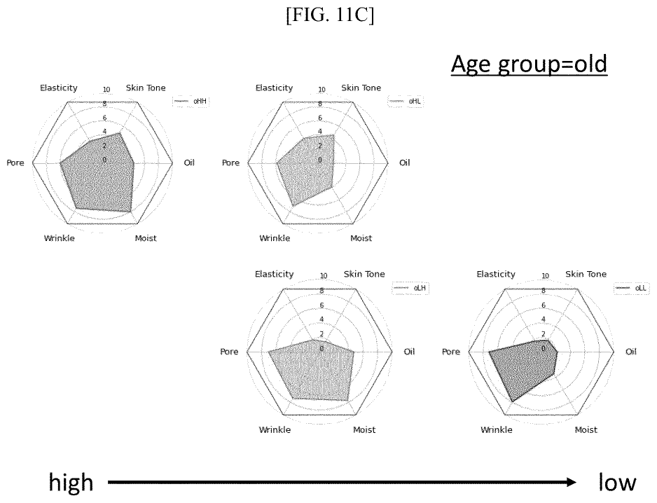

FIGS. 11a, 11b, and 11c illustrate a set of radial plots showing an exemplary set of skin types that may be used according to various embodiments.

In this specific example, clustering resulted in four distinct skin types. These types were determined using six skin parameters (elasticity, skin tone, oiliness, moisture, wrinkles, and pores) identified as the most or at least significantly important for determining skin type or skin quality. Additionally, depending on the embodiment, the radial plots may be generated based on age groups. Since skin condition is heavily dependent on age, it is desirable to compare a user's skin type with others in a similar age range. Comparing elderly users and younger users directly may be meaningless in terms of providing actionable information for skin improvement.

FIG. 11a corresponds to a younger age group, FIG. 11b to a middle-aged group, and FIG. 11c to an older age group. It can be observed that the evaluation indicators decrease from the left labeled “high” to the right labeled “low.” Radial plots that are separated into upper and lower regions in the middle area represent cases where some indicators are at a high level, while others are at a low level.

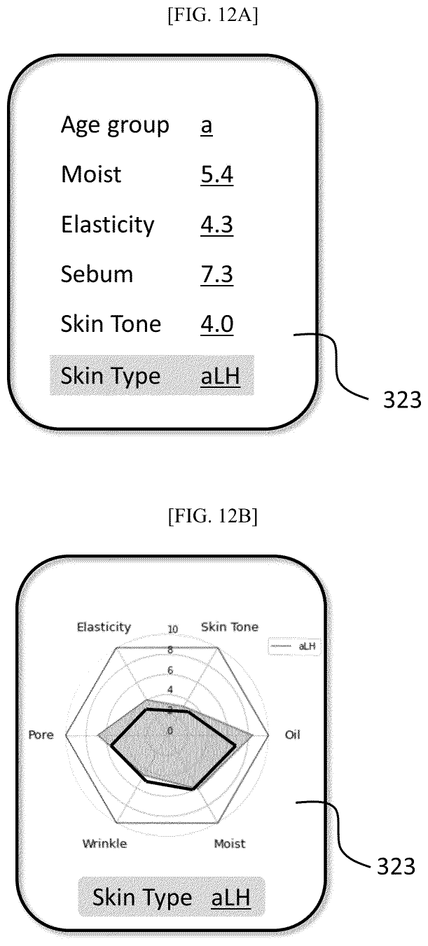

FIG. 12a displays the absolute skin score and phenotype on a display device (323), while FIG. 12b is a visualization for comparing values across different skin categories. This visualization allows users to compare and easily understand their skin phenotype or values for other skin types.

Calculating an absolute skin score regardless of age may be meaningless. Therefore, in some embodiments of the present invention, phenotype and visualized comparison graphs may be used to provide users with information about their skin type.

The age referenced in the present invention refers to the user's age at the time of measurement, based on their date of birth. The phenotype for a skin type may be indicated, for example, as “aLH,” meaning that both the user and the comparison target are in the middle-age group, with the first phenotype being on the lower side (“L”) and the second phenotype being on the higher side (“H”).

Without the ability to visually compare plots of these different types, users may not understand how to compare their data with that of other types. Furthermore, users can understand why their skin type differs from others through these comparisons. Users can also make comparisons within the same age group, providing them with relative information about their skin type. For example, a teenager comparing their skin type with that of a person in their 60s and concluding that their skin condition is excellent would be meaningless. In this sense, skin types according to at least one embodiment of the present invention provide accurate information that can help users of all ages improve their skin condition.

In addition to a plot displaying the user's own skin metric values, a plot showing the average values of users corresponding to a particular standard skin type-here, “aLH”—may also be provided. When these plots are displayed together, the user can understand why they have been classified as having a specific skin type by observing the general similarity in the shape of the plots. Furthermore, the user can grasp in what specific ways their skin compares to that of other users with the same skin type.

In another example, the display device (323) may provide a plot comparing the user's condition with that of an “aHH” type. The user can compare their skin condition with that of the top-tier skin type within the same age group. Based on such a comparison, the processor (321) may recommend improvement measures—such as cosmetics, medicines, or foods containing specific nutrients—to guide the user toward a more typical pattern.

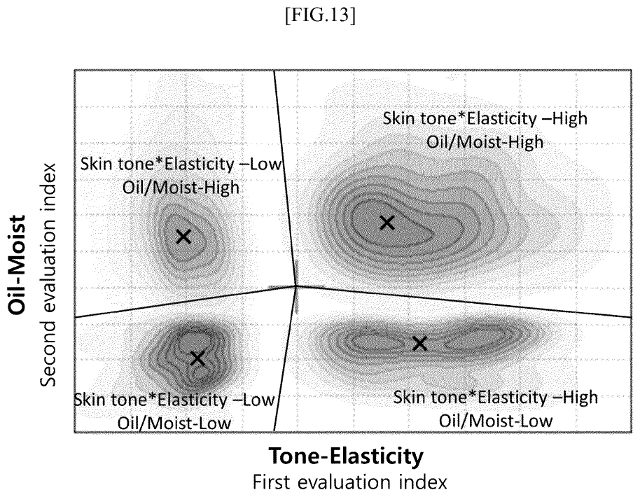

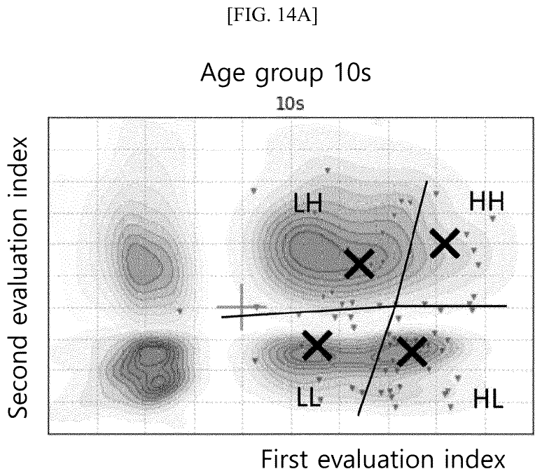

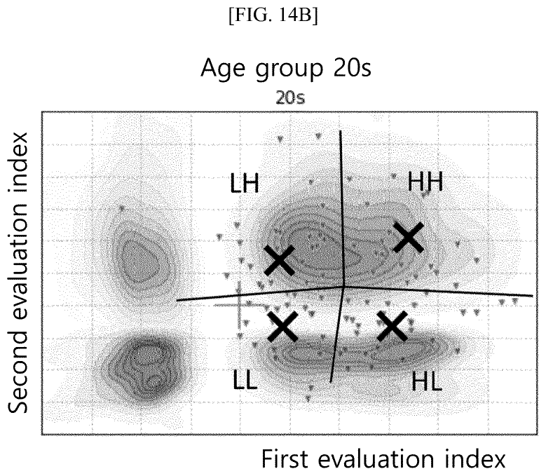

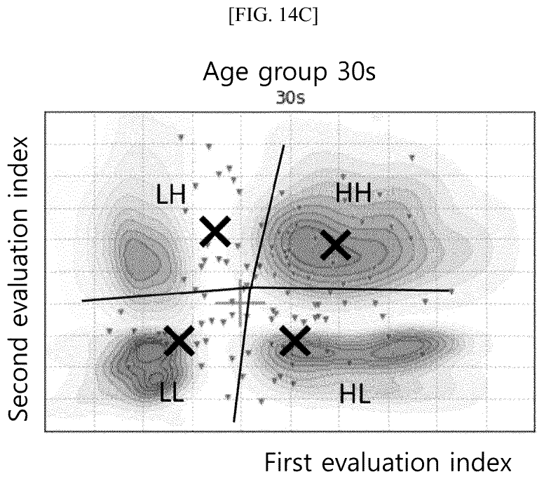

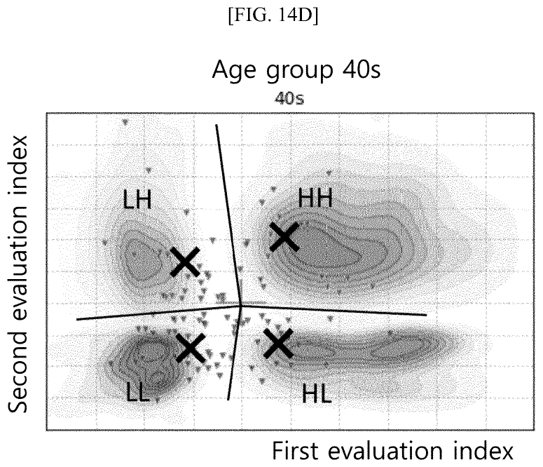

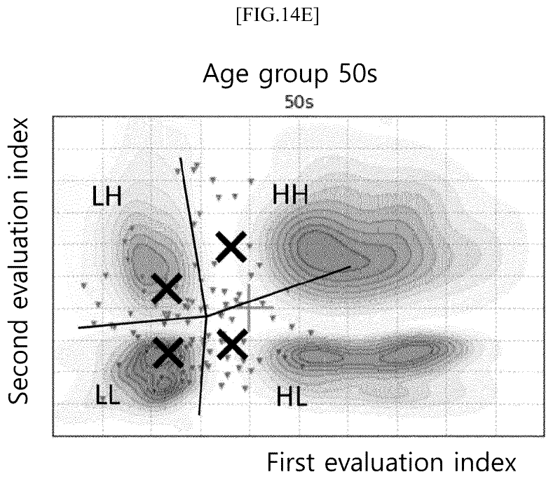

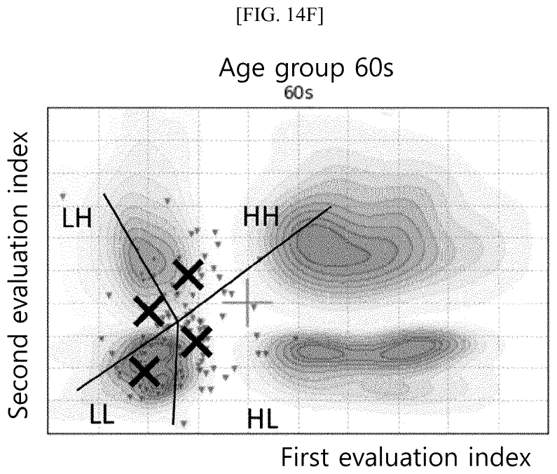

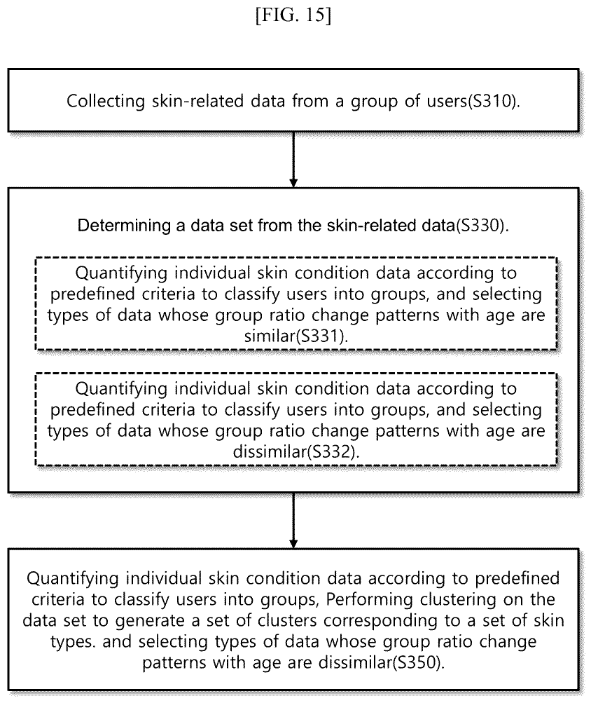

FIG. 13 illustrates an example in which skin types are categorized through clustering based on skin-related data from users across all age groups. FIGS. 14a through 14f show examples in which skin types are categorized through clustering by age group, in contrast to the classification in FIG. 13. FIG. 15 illustrates an exemplary process of generating clusters corresponding to sets of skin types that may be used according to various embodiments.

Referring to FIGS. 13, 14a through 14f, and 15, in order to determine a skin type according to various embodiments of the present invention, the method for identifying a skin type includes the following steps: collecting skin-related data from a group of users (S310); determining a data set of skin features from the skin-related data (S330); and performing clustering on the data set of skin features to generate a set of clusters corresponding to a set of skin types (S350). The clustering algorithm is k-means clustering, where k equals 4.

In a method for training a machine learning model for skin type identification performed by a computing device, the training method according to various embodiments of the present invention includes: collecting skin-related data from a group of users (S310); determining a data set of skin features from the skin-related data (S330); and performing clustering on the data set of skin features to generate a set of clusters corresponding to a set of skin types (S350).

The attributes of the data set are determined by quantifying individual skin condition data based on predefined criteria to divide them into groups, and by selecting types of data with similar patterns of proportional changes across age-based groups (S331).

The criterion for determining pattern similarity is the age range in which a reversal phenomenon occurs—i.e., when the ranking order of proportions among groups is reversed. Types of data with similar patterns include skin tone data and skin elasticity data.

The attribute of the data set is determined by normalizing the tone data and elasticity data and then performing a multiplication operation between them.

The attribute of the data set is determined by quantifying individual skin condition data based on predefined criteria to divide them into groups, and selecting types of data whose proportional change patterns by age group are dissimilar (S332).

The criterion for determining dissimilarity in patterns is whether the age range in which the reversal phenomenon occurs differs.

Another criterion for determining dissimilarity in patterns is whether the reversal phenomenon does not occur at all.

Another criterion for determining dissimilarity in patterns is whether the mismatch between the median and the mean exceeds a predetermined threshold.

Types of data with dissimilar patterns include sebum data and moisture data of the skin. The data set is generated by normalizing the skin's sebum data and moisture data, followed by a multiplication operation between them.

The attributes of the data set include information on skin color, skin elasticity, sebum, and moisture. The attributes include a first evaluation index obtained by multiplying skin color value with elasticity value, and a second evaluation index obtained by multiplying sebum value with moisture value.

The moisture value includes a calculation where the skin's hydration is divided by its transepidermal water loss (TEWL).

According to FIGS. 14a through 14f, it can be observed that the center of the spatial distribution moves from the upper right to the lower left as age increases.

Various embodiments of the present invention include the use of metrics such as Euclidean distance with centroid-based assignment, as used in the k-means clustering approach.

According to various embodiments of the present invention, when a data set divided by age group is used, the value of “K” is 4. The value of “K” may vary depending on whether age is included as an attribute.

Various clustering algorithms that can project selected features into a feature space using Euclidean distance and apply a k-means clustering approach may be employed.

In at least some embodiments, clustering may be optimized for aspects such as interpretability, conciseness of individual clusters, or separation between clusters.

In various embodiments, since clustering may perform poorly in high-dimensional spaces, approaches such as principal component analysis (PCA) may be used to select features and reduce dimensionality.

Then, a set of heuristics or rules may be applied to complete the skin types based on these clusters. Various distance heuristics may be used after clustering.

Distance metrics may include, in particular, Manhattan distance, Chebyshev distance, cosine similarity, Levenshtein or Hamming distance, Kendall-Tau distance, or weighted Manhattan distance.

In at least one embodiment, such distance metrics may take into account weighting based on the user's age.

Instead of representing all relevant features, a subset of these features (for example, six features) may be selected and displayed as the user's skin type data. These selected key features may correspond to advanced skin metrics, which are determined to be most important for evaluating and/or understanding the skin.

If a user may resemble two different skin types or does not have a dominant skin type association, at least some rules may be applied to select or adjust the displayed skin type for the user.

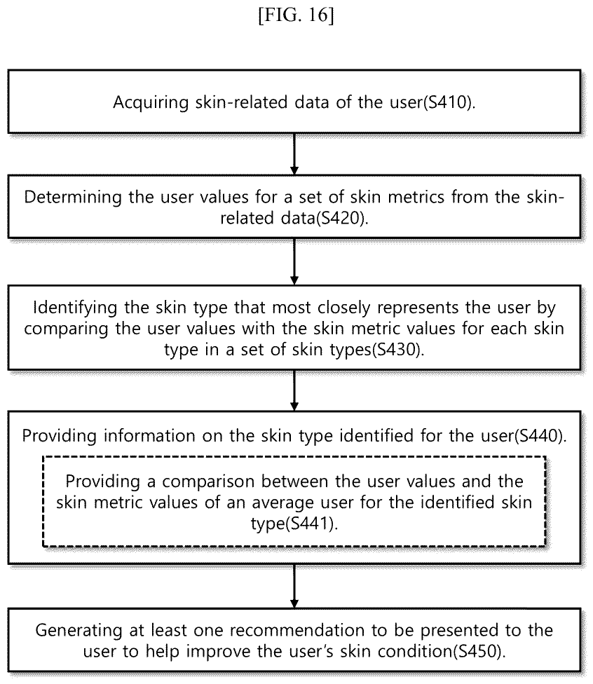

FIG. 16 illustrates an exemplary process for determining a user's skin type and providing information for skin improvement, which may be used in various embodiments.

A skin type identification method according to various embodiments of the present invention, performed by a computing device, includes: acquiring user skin-related data (S410); determining user values for a set of skin metrics from the skin-related data (S420); comparing the user values to the skin metric values for each skin type in a set of skin types to identify the skin type that most closely represents the user (S430); and providing information about the identified skin type to the user (S440).

The step of providing information includes providing a comparison between the user's skin metric values and the average skin metric values of users for the identified skin type (S441).

The skin types are classified into a first skin type and a second skin type. The user's skin-related data corresponding to the skin metric for identifying the first skin type includes a value obtained by multiplying the user's skin tone by the user's skin elasticity.

The skin types are classified into a first skin type and a second skin type. The user's skin-related data corresponding to the skin metric for identifying the second skin type includes a value obtained by multiplying the user's skin oiliness value by the user's skin moisture value.

The moisture value includes a value obtained by dividing the skin's moisture content (hydration) by the skin's transepidermal water loss (TWML). The moisture data obtained by the measurement system includes moisture content and moisture loss, and the moisture data is the value of moisture content divided by moisture loss.

The skin's moisture content and moisture loss may be measured by a moisture sensor (140) as shown in FIG. 1, or by moisture sensors (314, 427) of the measurement system described later.

A processor (321) or a service provider system (350) collects skin-related data from multiple users. In such examples, skin-related data for a user may be collected once, daily, over multiple days, or over multiple months.

Data collection and generation may be performed in a main device (320) that receives and processes one or more data from a sub-device (310) having at least one sensor, or from multiple sub-devices (310).

In at least some embodiments, data processing and skin type determination may be performed by the service provider system (350).

A skin type identification method according to various embodiments of the present invention includes identifying the user's skin type using one or more distance heuristics by comparing the user values with each skin metric value of a set of clusters. Values within a defined range are between 0 and 1, and one or more programs multiply a first age weight based on the user's age by a first evaluation index to determine a first phenotype, and store the first phenotype and a second phenotype in memory (327).

In at least some embodiments, the sub-device (310), the main device (320), or the service provider system (350) may perform data preprocessing such as noise removal, duplicate data removal, selection of relevant data, and normalization. The data may originate from multiple sources at multiple locations.

The processor (321) may determine values for a set of skin metrics for the user using at least some of the collected data.

These may include skin metrics determined to be particularly important for skin analysis (e.g., tone, elasticity, pores, wrinkles, oiliness, moisture, first evaluation index, second evaluation index, first age weight, second age weight).

Then, the user skin metric values as well as other potential metrics may be compared to values of a set of clusters, each of which is associated with a skin type (e.g., a skin phenotype).

The processor (321) or the service provider system (350) may determine one of these skin types using one or more distance heuristics or another such approach.

The skin type identification method according to various embodiments of the present invention includes generating at least one recommendation to be presented to the user to help improve the user's skin (step S450), wherein the at least one recommendation is determined at least partially based on the user values for the skin metrics and the identified skin type for the user.

In such an example, information and visualization about the selected user's skin type may be provided regarding individual variances from average or expected values of users with that skin type, as well as information for skin type improvement.

In an exemplary embodiment, the information may include skin improvement solutions, preferred lifestyle habits, dietary habits, or recommended cosmetics containing specific ingredients.

Generally, through machine learning techniques (extensions), one or more systems may be trained with sets of metrics, skin-related data, and data for specific users and/or general populations.

Then, skin-related data and other metrics are captured from a particular user and analyzed by the trained system to determine relationships among the user's metrics, after which the skin type and related information may be pushed to the user.

For example, a machine learning system using a convolutional neural network (CNN) may be designed to extract metrics from the aforementioned skin-related data. The convolutional neural network may be trained on skin-related datasets and determine optimal skin types by age.

Long short-term memory neural networks (LSTM), hidden Markov models, or other time series models may be designed to predict state events based on prior history, and such models may also consider any of the appropriate variables discussed herein.

In some embodiments, deep neural networks or other machine learning approaches may be used to “learn” patterns based on metrics obtained from such information.

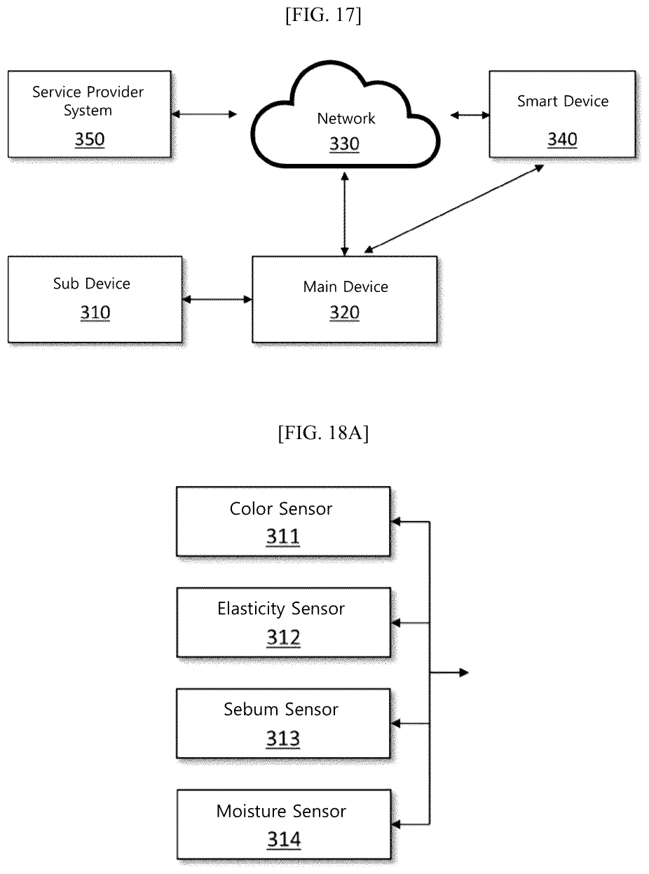

FIG. 17 illustrates an overview of a system according to various embodiments of the present invention.

Referring to FIG. 17, in at least one embodiment, skin-related data may be collected or determined using a sub-device (310) and/or a main device (320).

The sub-device (310) and the main device (320) may include sensors and a processor (321) capable of collecting or determining skin-related data, and may provide or receive such data to or from other computing devices via one or more networks (330) across one or more other devices or channels. The sub-device (310) includes a device capable of acquiring and providing skin-related data.

In at least one embodiment, such data may be collected for analysis by the main device (320) executing an application having skin analysis functionality.

In some embodiments, the skin-related data may be analyzed by a user computing device, such as a desktop computer.

In at least some embodiments, at least some of this data may be provided to the sub-device (310) or a service provider system (350) associated with skin monitoring software.

In some embodiments, users may subscribe to services provided by the service provider system (350), which may receive data and provide analyses of the skin-related data or information for skin improvement.

The method by which the service provider system (350) connected via network (330) aggregates and analyzes skin-related data to determine various skin-related metrics and information has several advantages.

Most data analysis and processing may be performed by remote systems, services, or devices. This also reduces the data throughput or memory (327) capacity burden on the sub-device (310) and the main device (320) used to collect and transmit the data.

Additionally, the service provider may collect data from other users, enabling more accurate comparisons with other users. Results of skin condition analyses may be provided back to users or related systems, devices, services, or providers. Data or commands constituting judgment results and recommendations may be provided to the main device (320) or a smart device (340) designated by the user.

In some embodiments, a device having an interface may ask the user questions with simple yes/no answers.

In some embodiments, the device may design or conduct surveys regarding users' age, lifestyle habits, skin improvement goals, and skin conditions.

Any one or all of this information may be used to adjust weights of one or more metrics, which allows the user to align more closely or reliably with a particular skin type.

Measurement systems according to various embodiments of the present invention capture skin-related data.

The captured skin-related data may include, but is not limited to, any potentially relevant data such as skin color, skin elasticity, trans-epidermal water loss (TEWL), skin hydration, skin oil or sebum or collagen content, number of wrinkles, and number of pores.

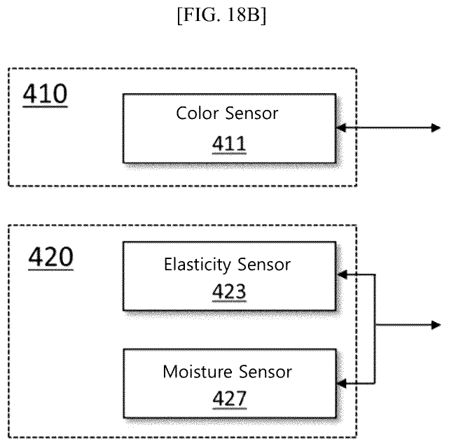

FIG. 18a is a block diagram of various embodiments of the sub-device (310) of FIG. 14, and FIG. 18b is a block diagram of another embodiment of the sub-device (310) of FIG. 14.

Referring to FIG. 18a, the sub-device (310) includes a color sensor (411), an elasticity sensor (423), an oil sensor, and a moisture sensor (427). The sub-device (310) transmits skin-related data to the main device (320) or the service management system.

Referring to FIG. 18b, the sub-device (310) includes a first sub-device (410) including a color sensor (411) and a second sub-device (420) including an elasticity sensor (423) and a moisture sensor (427).

The first sub-device (410) transmits skin tone data and skin oil data. The color sensor (411) of the first sub-device (410) transmits skin tone data and oil data.

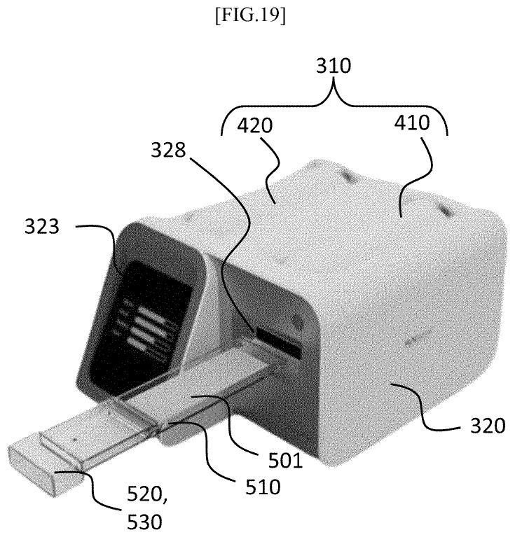

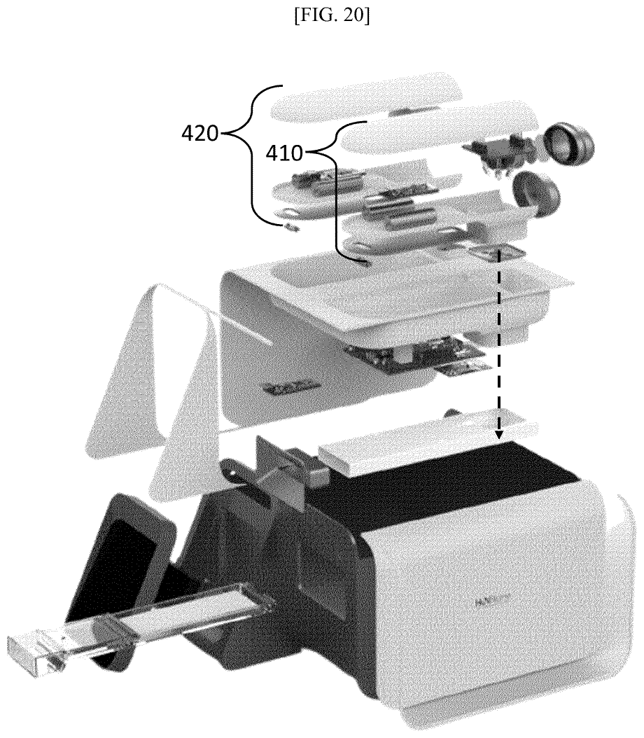

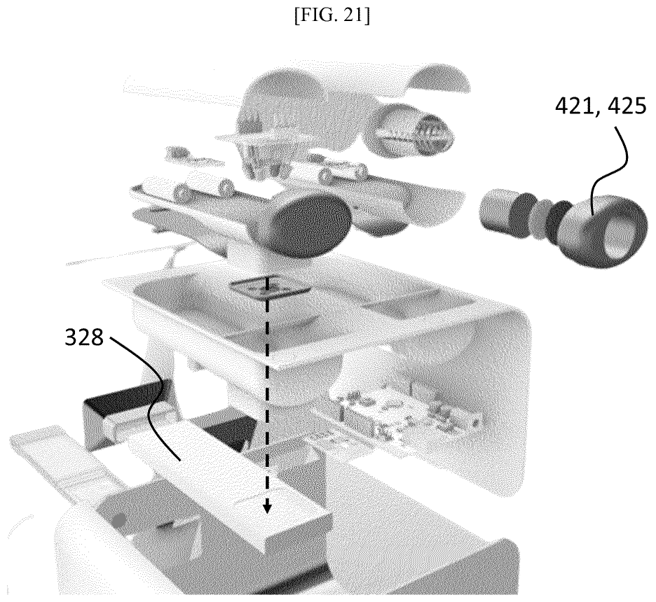

FIG. 19 is a perspective view of a skin measurement system according to various embodiments of the present invention. FIG. 20 is an exploded perspective view of the skin measurement system of FIG. 19. FIG. 21 is a rear view of the perspective view of FIG. 20.

Referring to FIGS. 19 to 21, the skin measurement system according to various embodiments of the present invention includes a first sub-device (410), a second sub-device (420), and a main device (320).

The main device (320) forms a slot (328) into which a container (510) is inserted. The film (501) contains sebum or oil collected from the skin. The film (501) is accommodated in the container (510).

The light source (413) and color sensor (411) of the first sub-device (410) are arranged facing the film (501) accommodated in the slot (328). The main device (320) includes a display device (323). The display device (323) may use a smart device (340) of the user account.

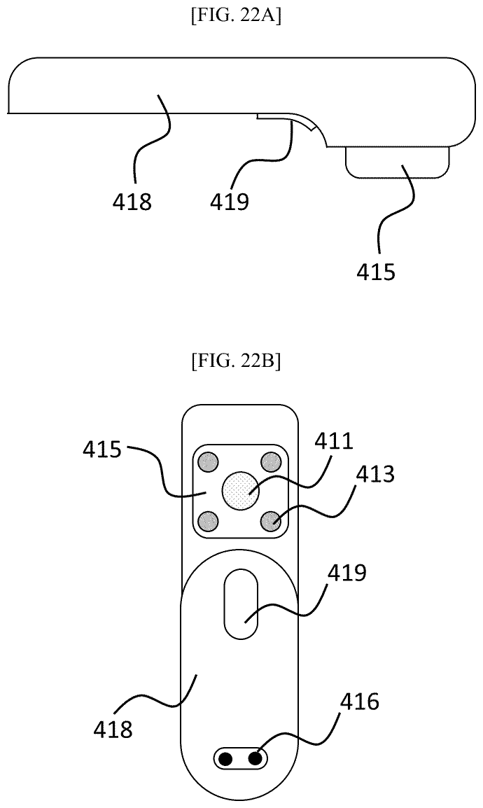

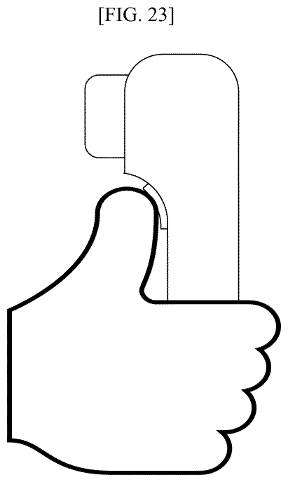

FIG. 22a is a side view of the first sub-device (410) according to various embodiments of the present invention. FIG. 22b is a bottom view of the first sub-device (410) according to various embodiments of the present invention. FIG. 23 illustrates a user gripping the handle portion (418) and pressing the button (419).

The first sub-device (410) according to various embodiments of the present invention includes a head portion (415) accommodating the color sensor (411) and the light source (413); a handle portion (418) connected to the head portion (415) and curved at one side; and a button (419) formed at the curved part of the handle portion (418) with the same curvature as the handle portion (418).

The button (419) is formed with the same curvature as the position of the handle portion (418) where the button (419) is inserted. This shape corresponds to the shape of the user's thumb, reducing discomfort and minimizing the probability of malfunction due to the identical curvature.

The first sub-device (410) includes terminals electrically connected to the main device (320). The first sub-device (410) includes at least one light source (413) formed around the color sensor (411) to irradiate light through a receiving hole (329).

FIG. 24a shows inserting the film (501) into the container (510); FIG. 24b shows staining the film (501) with a staining reagent (521); FIG. 24c shows washing off the staining reagent (521) with a washing buffer (531); and FIG. 24d shows the film (501) after staining is completed.

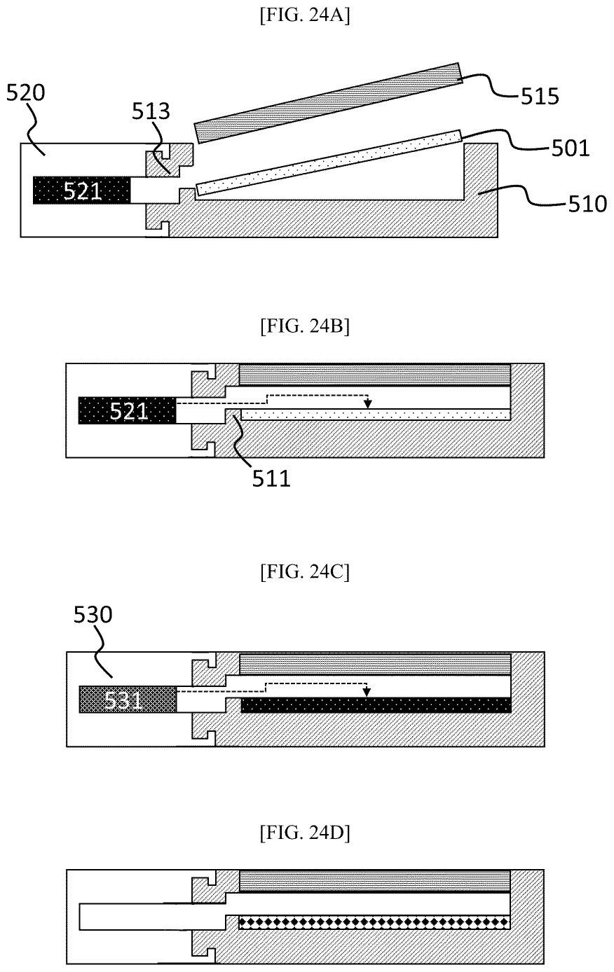

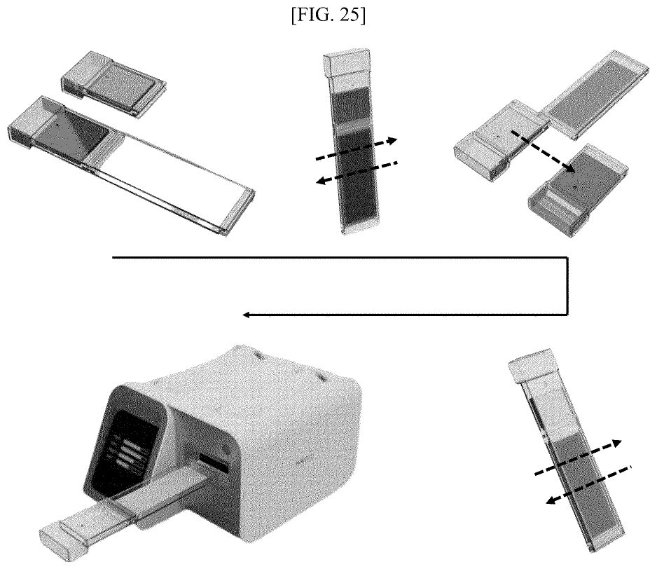

FIG. 25 illustrates the usage process of FIG. 24.

The measurement system according to various embodiments of the present invention includes a container (510) accommodating the film (501) and inserted into the slot (328); a staining cartridge (520) detachable from the container (510) and containing ORO (Oil Red O); and a washing cartridge (530) detachable from the container (510) and containing the washing buffer (531).

The container (510) includes a cover (515). The cover (515) is detachably coupled to the container (510). The container (510) and the cover (515) may be made of a glass material. The container (510) and the cover (515) may be formed of a transparent polymer compound that is not stained by coloring agents, such as “ORO.” The container (510) and the cover (515) may also be made of plastic.

First, the user collects sebum from the skin using the film (501). The user opens the cover (515) and inserts the film (501) into the container (510). The user shakes the container (510) together with the staining cartridge (520), which contains the staining reagent (521), to stain the sebum.

A protruding diffusion part (511) is formed in the container (510). The diffusion part (511) functions to change the flow path so that the staining reagent (521) is uniformly absorbed by the film (501). The diffusion part (511) protrudes at the inlet where the staining reagent (521) flows in. The diffusion part (511) is formed protruding between the film (501) and the inlet. The diffusion part (511) may be formed inclined such that its height increases from the inlet toward the film (501).

The user removes the staining cartridge (520) and attaches the washing cartridge (530) to the container (510). The staining cartridge (520) and the washing cartridge (530) are formed with coupling rails (513) that slidably couple with the container (510).

The staining cartridge (520) and the washing cartridge (530) can be fastened or detached by moving them laterally relative to the container (510). The washing buffer (531) removes any staining reagent (521) that has not bound to the sebum.

In various embodiments of the present invention, the staining reagent (521) is ORO (Oil Red O).

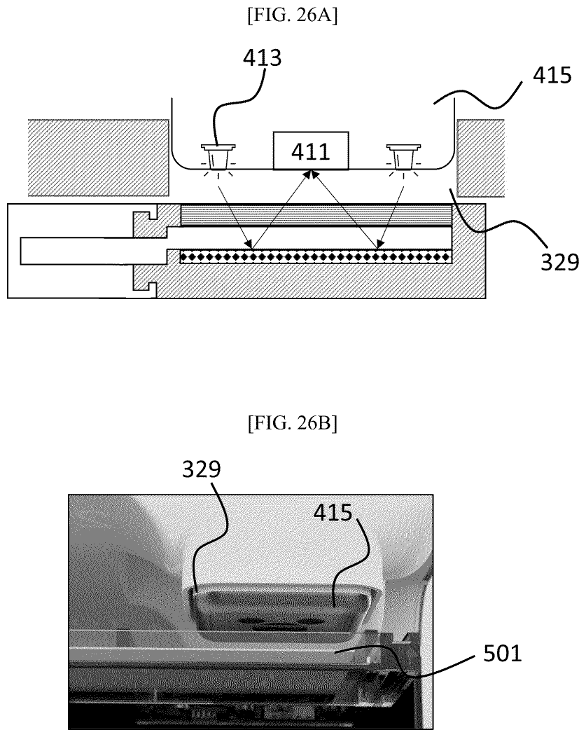

FIG. 26a illustrates the acquisition of an optical signal passing through the receiving hole (329) by the color sensor (411), and FIG. 26b illustrates the first sub-device (410) passing through the receiving hole (329) inside the main device (320).

Referring to FIGS. 26a and 26b, the skin measurement system according to various embodiments of the present invention includes: a first sub-device (410) including a color sensor (411) that receives an optical signal; and a main device (320) having a receiving hole (329) that forms a path for the optical signal incident on the color sensor (411), and a slot (328) into which a film (501) collecting sebum from the user's skin is inserted, the slot (328) being formed to extend to the receiving hole (329).

Some of the light irradiated from the light source (413) is absorbed by the film (501). The film (501) reflects some of the light. The color sensor (411) acquires the optical signal. The processor (321) analyzes the optical signal to calculate the amount of sebum.

In various embodiments, the emitter (or light source (413)) includes an electronic semiconductor light source such as an LED or uses any of filaments, phosphors, or lasers to generate light.

The color sensor (411) may include any one of a photodiode, phototransistor, charge-coupled device (“CCD”), or complementary metal-oxide-semiconductor (“CMOS”) sensor.

In various embodiments, the color sensor (411) may be mounted in a housing having one or more filters configured to filter wavelengths of light other than those emitted by the light source (413).

Although LEDs and photodiodes are used as examples of the light sources (413) and optical color sensors, respectively, the technologies described in the present invention can be extended to other types of light sources, such as edge-emitting lasers, surface-emitting lasers, and LED-pumped phosphors that generate broadband light.

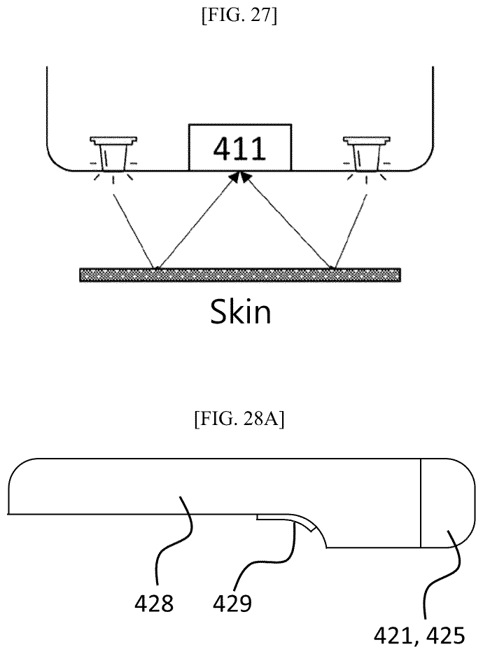

FIG. 27 illustrates separating the first sub-device (410) from the main device (320) to measure the skin tone.

The first sub-device (410) is detachably coupled to the main device (320), and when detached from the main device (320), the received signals are interpreted as skin tone data. The first sub-device (410) is provided with terminals that electrically connect to the main device (320). The terminals allow current and/or signals to connect with the main device (320).

The processor (321) can switch to a mode for measuring skin tone when the main device (320) and the first sub-device (410) are electrically separated. The processor (321) can switch to a mode for measuring sebum on the film (501) when the main device (320) and the first sub-device (410) are electrically connected. The processor (321) can determine the connection or separation through the terminals formed on the first sub-device (410).

Light emitted from one or more light sources (413) may be reflected back from the skin to the color sensor (411). The color sensor (411) acquires the optical signal from the skin and can determine the skin tone.

The processor (321) can distinguish between the skin tone measurement mode and the sebum measurement mode and label the measured data accordingly.

The main device (320) includes a display device (323); at least one processor (321); and a memory (327) storing instructions which, when executed by the at least one processor (321): cause the device to acquire user skin-related data using the color sensor (411); determine user values for a set of skin metrics from the skin-related data; compare the user values to skin metric values for each of a set of skin types; identify the skin type that most closely represents the user's skin; and display information about the identified skin type on the display device (323).

The skin-related data comprises sebum data of the user's skin and skin tone data of the user's skin.

A method of implementing a skin measurement system according to various embodiments of the present invention includes: a first sub-device (410) including a color sensor (411) configured to receive optical signals; and a main device (320) having a receiving hole (329) forming an optical path for light incident on the color sensor (411) and a slot (328) into which a film (501) collecting sebum from the user's skin is inserted, the method comprising: obtaining sebum data and skin tone data of a user's skin from the first sub-device (410) (S410); determining user values for a set of skin metrics from the skin-related data (S420); comparing the user values to skin metric values for each skin type in a set of skin types and identifying the skin type that most closely represents the user's skin (S430); and providing information about the identified skin type to the user (S440).

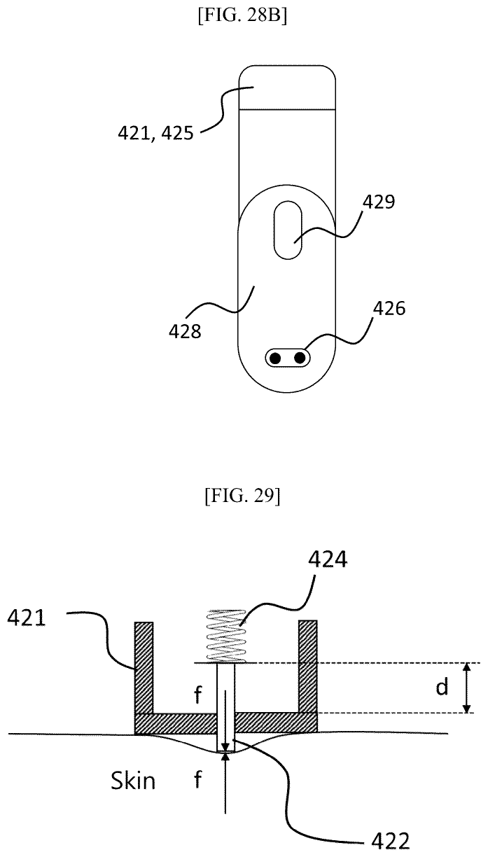

FIG. 28a is a side view of a second sub-device (420) according to various embodiments of the present invention, and FIG. 28b is a bottom view of the second sub-device (420).

The second sub-device (420) includes: a first cap (421) having protrusions (422) protruding therefrom; and a handle portion (428) detachably coupled to the cap. A button (429) having the same curvature as the curved portion of the handle portion (428) is formed on the curved portion of the handle portion (428). Terminals are formed on the handle portion (428).

FIG. 29 illustrates the second sub-device (420) according to various embodiments of the present invention measuring elasticity.

The skin measurement system according to various embodiments of the present invention includes: the second sub-device (420) including protrusions (422) contacting the skin and an elasticity sensor (423) measuring a force by which the skin pushes back the protrusions (422) or displacement (d) of the protrusions (422); and a main device (320) electrically connected to the second sub-device (420), acquiring data related to skin elasticity and displaying information about the user's skin condition.

A user contacts the second sub-device (420) to the skin. The skin pushes the protrusions (422), and the protrusions (422) push back the skin. The same force (f) acts at this time. The elasticity sensor (423) measures the displacement (d) of the protrusions (422) to measure the force (f). If an elastic member (424) pushes the protrusions (422), the elasticity sensor (423) may measure the displacement (d) of the protrusions (422). The processor (321) can calculate the force (f) using the displacement (d) of the protrusions (422) and the elastic modulus of the elastic member (424).

In another example, the elasticity sensor (423) may directly measure the force (f) applied to the protrusions (422). The elasticity sensor (423) may be a strain gauge. The elasticity sensor (423) may be a load cell.

The protrusions (422) may have a large diameter shape. Such a shape has the advantage of causing less irritation to the skin. The protrusions (422) in the present invention should not be interpreted as limited by the relative ratio of diameter to length.

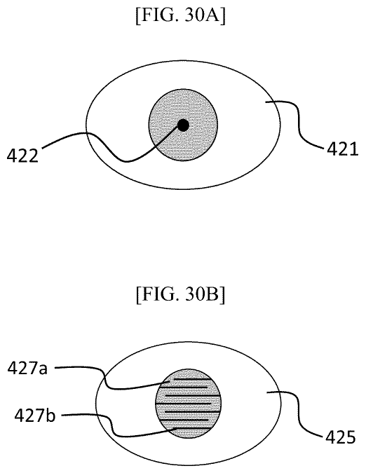

FIG. 30 illustrates front views of the first cap (421) and the second cap (425) according to various embodiments of the present invention.

The skin measurement system according to various embodiments of the present invention includes: a second sub-device (420) including a moisture sensor (427) comprising comb-shaped electrodes (427a) contacting the skin and a conductive pad (427b) absorbing moisture from the skin and measuring electrical resistance to determine moisture content; and a main device (320) electrically connected to the second sub-device (420), acquiring data related to skin moisture and displaying information about the user's skin condition.

The skin measurement system according to various embodiments of the present invention includes a first cap (421) comprising a protrusion (422) that contacts the skin and an elasticity sensor (423) that measures the force with which the skin pushes back the protrusion (422) or the displacement (d) of the protrusion (422). The first cap (421) and the second cap (425) are detachably coupled to the main device (320) so that they are interchangeable with each other.

The second sub-device (420) includes a second cap (425) having a moisture sensor (427) formed of electrodes (427a) and a conductive pad (427b); and a handle portion (428) to which the cap is detachably coupled. The second sub-device (420) includes the second cap (425) that is formed with comb-shaped electrodes (427a) contacting the skin and a conductive pad (427b) that absorbs moisture from the skin. The first cap (421) and the second cap (425) are detachably coupled to the main device (320) so as to be interchangeable. The second sub-device (420) acquires at least two types of data by exchanging the first cap (421) and the second cap (425).

The skin measurement system according to various embodiments of the present invention includes: a second sub-device (420) that comprises an elasticity sensor (423) that measures the force by which the skin pushes back the protrusion (422) contacting the skin or the displacement (d) of the protrusion (422); and a moisture sensor (427), which measures the moisture contained in the skin by measuring electrical resistance, the moisture sensor (427) comprising comb-shaped electrodes (427a) contacting the skin and a conductive pad (427b) that absorbs moisture from the skin; and a main device (320) electrically connected to the second sub-device (420), configured to acquire data related to skin elasticity and skin moisture and display information about the user's skin condition.

The main device (320) includes a display device (323) configured to display relevant information; at least one processor (321); and a memory (327) storing instructions. When the instructions are executed by at least one processor (321), the main device: acquires user-related skin moisture data and skin elasticity data; determines user values for a set of skin metrics based at least on the skin-related data; and compares the user values with skin metric values for each skin type in a set of skin types to identify the skin type that most closely represents the user's skin.

The second sub-device (420) includes: a cap including either the elasticity sensor (423) or the moisture sensor (427); a handle portion (428) connected to the cap and curved at one end; and a button (429) formed on the curved portion of the handle portion (428) with a curvature equal to that of the handle portion (428).

The method for implementing the skin measurement system according to various embodiments of the present invention includes: a second sub-device (420) comprising an elasticity sensor (423) that measures the force with which the skin pushes back the protrusion (422) contacting the skin or the displacement (d) of the protrusion (422); and a moisture sensor (427) comprising comb-shaped electrodes (427a) contacting the skin and a conductive pad (427b) that absorbs moisture from the skin, measuring the moisture contained in the skin by measuring electrical resistance; and a main device (320) electrically connected to the second sub-device (420), configured to acquire data relating to skin elasticity and skin moisture and display information about the user's skin condition. The implementation method comprises acquiring user skin elasticity data and moisture data from the second sub-device (Step S410), determining user values for a set of skin metrics based on the acquired skin-related data (Step S420), comparing the determined user values with skin metric values for each skin type in a set of skin types to identify the skin type that most closely represents the user's skin (Step S430), and providing information about the identified skin type to the user (Step S440).

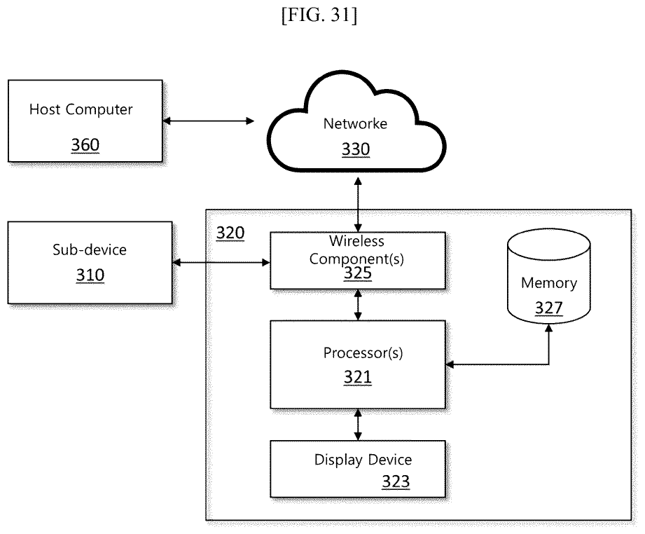

FIG. 31 illustrates components of an exemplary computing device that may be used according to various embodiments.

In such an example, the main device (320) includes a memory (327), which may include flash memory or DRAM, and at least one processor (321) such as a central processing device (“CPU”) or a graphics processing device (“GPU”) configured to execute instructions stored in the memory (327).

Referring to FIG. 31, the exemplary monitoring device may include one or more processors (321) coupled to memory (327), a display, a bus, one or more input/output (I/O) elements, and wireless networking components (325), among other possible options.

The processor (321) may calculate values for skin-related metrics based on reflected light signals generated by the color sensor (411).

In various embodiments, data detected by each sensor may be transmitted to the main device (320) and/or a host computer (360) over at least one network (330) via near-field communication (“NFC”), Bluetooth, Wi-Fi, or other suitable wireless communication protocols for analysis, display, reporting, or other such uses.

The memory (327) may include RAM, ROM, flash memory, or other non-volatile digital data storage and may include control programs comprising sequences of instructions which, when loaded from the memory (327) and executed by the processor (321), cause the processor (321) to perform functions described in the present invention.

The light sources (413) and sensors may be coupled directly or indirectly to the bus via driver circuits, by which the processor (321) drives the light sources (413) and acquires signals from the sub-device (310) and various sensors.

The host computer (360) may communicate with the wireless networking components (325) via one or more networks (330), which may include one or more local area networks (330), wide area networks (330), and/or the Internet using any terrestrial or satellite links. In some embodiments, the host computer (360) executes control programs and/or applications configured to perform some of the functions described in this invention.

The main device (320) also includes wireless components (325) operable to communicate with the sub-device (310) and/or one or more electronic devices within the communication range of a specific wireless channel.

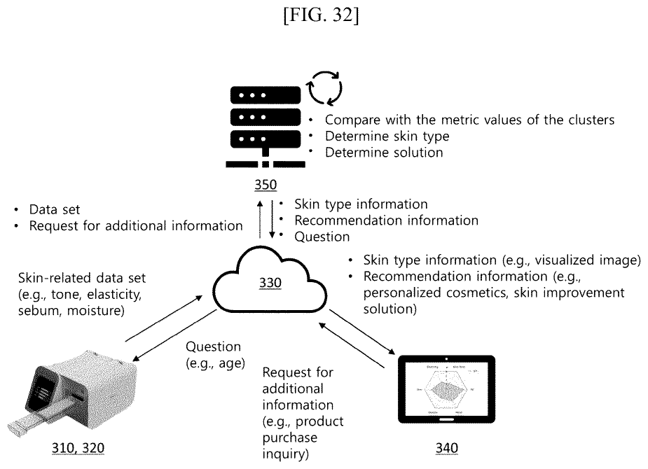

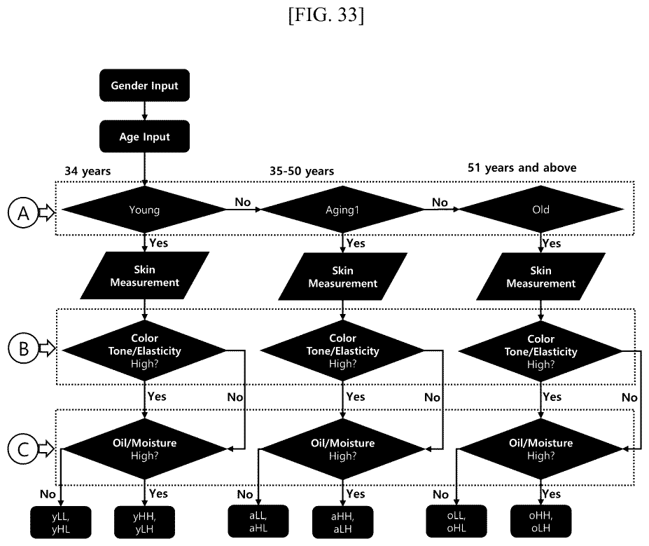

FIG. 32 is a diagram illustrating the transmission of data provided according to various embodiments.

Referring to FIG. 32, the sub-device (310) and the main device (320) transmit skin-related data to a service provider system (350) via a network (330). The skin-related data includes data related to skin tone, moisture, oil content, and elasticity. The main device (320) can display content instructed by the service provider system (350). For example, the main device (320) may ask the user for their age.

The service provider system (350) determines the skin type by comparing the input skin-related data with metric values of clusters. The service provider system (350) transmits the determined skin type and the determined skin improvement solutions over the network.

The user can view their skin information using a smart device (340) and can also compare it with other users of the same age group. The user may receive information regarding skin improvement solutions. The user can request additional information via the network. For example, the user may request subscription information for skin improvement solutions, product purchase information, and instructions on how to use the products.

The service provider system (350) can receive skin-related data transmitted from multiple users, and the collected data can be utilized for clustering related to skin types and for machine learning of skin improvement solutions.

Although the preferred embodiments of the present invention have been described and illustrated above, the present invention is not limited to the specific embodiments described above. Without departing from the scope of the claims, various modifications and embodiments that can be implemented by those skilled in the art to which the invention pertains are possible. Such modifications and embodiments should not be understood individually apart from the technical spirit or prospects of the present invention.

1. A method for training a machine learning model for identifying skin types, the method comprising:
collecting skin-related data for a group of users;
determining a data set of skin features based on the collected skin-related data; and
performing clustering on the data set of skin features to generate a set of clusters corresponding to a set of skin types.

2. The method of claim 1, wherein
the attributes of the data set are determined by quantifying individual skin condition data based on predetermined criteria to classify into groups, and selecting types of data that exhibit similar variation patterns in group proportions according to age.

3. The method of claim 2, wherein
the criterion for determining the similarity of the patterns is an age group in which an inversion phenomenon occurs in the ranking of group proportions.

4. The method of claim 2, wherein
the types of data with similar patterns comprise skin tone data and skin elasticity data.

5. The method of claim 2, wherein
the attributes of the data set are determined by normalizing the skin tone data and the skin elasticity data, and performing a multiplication operation between them.

6. The method of claim 1, wherein
the attributes of the data set are determined by quantifying individual skin condition data based on predetermined criteria to classify into groups, and selecting types of data that exhibit dissimilar variation patterns in group proportions according to age.

7. The method of claim 6, wherein
the criterion for determining the dissimilarity of the patterns is that the age groups in which an inversion in the ranking of group proportions occurs are different.

8. The method of claim 6, wherein
the criterion for determining the dissimilarity of the patterns is whether or not such an inversion phenomenon exists.

9. The method of claim 6, wherein
the criterion for determining the dissimilarity of the patterns is whether the difference between a median value and a mean value exceeds a predetermined threshold.

10. The method of claim 6, wherein
the types of data exhibiting dissimilar patterns comprise skin oil data and skin moisture data,
and the data set is determined by normalizing the skin oil data and the skin moisture data, and performing a multiplication operation between them.

11. A measurement system comprising:
a sub-device including a color sensor configured to receive an optical signal; and
a main device including a receiving hole configured to form a path of the optical signal incident on the color sensor, and a slot in which a film having user skin oil collected thereon is inserted,
wherein the slot extends to the receiving hole.

12. The measurement system of claim 11, wherein
the film is stained with Oil Red O (ORO).

13. The measurement system of claim 11, further comprising:
a container configured to accommodate the film and to be inserted into the slot; and
a staining cartridge, detachably connectable to the container, and configured to accommodate a staining reagent.

14. The measurement system of claim 11, further comprising:
a container configured to accommodate the film and to be inserted into the slot; and
a washing cartridge, detachably connectable to the container, and configured to accommodate a washing buffer.

15. The measurement system of claim 11, wherein
the sub-device further comprises at least one light source formed around the color sensor and configured to irradiate light into the receiving hole.

16. The measurement system of claim 11, wherein
the main device comprises:
a display device;
at least one processor; and
a memory storing instructions,
wherein the instructions, when executed by the at least one processor, cause the processor to:
acquire user skin-related data using the color sensor;
determine user values for a set of skin metrics from at least the skin-related data;
identify a skin type that most closely represents the user's skin by comparing the user values with skin metric values for each of a set of skin types; and
display information regarding the identified skin type of the user on the display device.

17. The measurement system of claim 16, wherein
the skin-related data comprises user skin oil data and user skin tone data.

18. The measurement system of claim 17, wherein
the sub-device is detachably coupled to the main device, and
a signal received while the sub-device is detached from the main device is determined to be skin tone data.

19. The measurement system of claim 11, wherein
the sub-device comprises:
a head portion accommodating the color sensor;
a handle portion connected to the head portion and having a curved side; and
a button formed on the curved portion of the handle portion with a curvature matching that of the handle portion.

20-29. (canceled)

30. A method for implementing a measurement system comprising:
an elasticity sensor that measures a force by which the skin pushes a protrusion or a displacement of the protrusion contacting the skin;
a moisture sensor comprising a comb-shaped electrode contacting the skin and a conductive pad absorbing skin moisture, and measuring an electrical resistance to determine moisture content in the skin;
a sub-device including the elasticity sensor and the moisture sensor; and
a main device electrically connected to the sub-device, configured to acquire data related to skin elasticity and skin moisture and display information about the user's skin condition, the method comprising:
acquiring skin elasticity data and moisture data of a user from the sub-device;
determining user values for a set of skin metrics from the skin-related data;
identifying a skin type that most closely represents the user's skin by comparing the user values with skin metric values corresponding to each of a set of skin types; and
providing information about the identified skin type of the user.
